(19) United States
(12) Patent Application Publication
(10) Pub. No.: US 2015/0366946 A1
(43) Pub. Date: Dec. 24, 2015

(54) PHARMACEUTICAL COMPOSITIONS FOR ORAL TREATMENT OF DIABETES

(71) Applicant: OSHADI DRUG ADMINISTRATION LTD., Rehovot (IL)

(72) Inventors: Alexander Vol, Rehovot (IL); Orna Gribova, Rehovot (IL)

(21) Appl. No.: 14/763,016

(22) PCT Filed: Jan. 29, 2014
(86) PCT No.: PCT/IL2014/050099

§ 371 Date: Jul. 23, 2015

Related U.S. Application Data
(60) Provisional application No. 61/880,996, filed on Sep. 23, 2013.
(60) Provisional application No. 61/757,762, filed on Jan. 29, 2013.

(51) Int. Cl. A61K 38/28; A61K 47/36; A61K 9/28; A61K 47/44; A61K 45/06; A61K 9/00; A61K 47/02

(52) U.S. Cl. CPC A61K 38/28; A61K 9/0053; A61K 47/36; A61K 47/02; A61K 47/44; A61K 45/06; A61K 9/28

(57) ABSTRACT

The present invention relates to pharmaceutical compositions for oral delivery comprising at least two bioactive proteins associated with glucose metabolism, selected from the group consisting of insulin, proinsulin and C-Peptide in a delivery vehicle adapted for oral administration that provides portal delivery of bioactive proteins. The exemplary pharmaceutical compositions comprise an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with the at least two bioactive proteins. The present invention further provides therapeutic uses of said pharmaceutical compositions.

### FIELD OF INVENTION

The present invention relates to pharmaceutical compositions for oral delivery of combinations of at least two bioactive proteins associated with glucose metabolism, selected from the group consisting of insulin, proinsulin and C-peptide. The pharmaceutical compositions of the invention provide portal delivery of the bioactive proteins. The present invention further provides therapeutic uses of said pharmaceutical compositions.

### BACKGROUND OF THE INVENTION

Insulin is the mainstay of treatment for people with type I diabetes. Insulin is also an important treatment tool for people with type II diabetes, when their blood glucose levels cannot be controlled by diet, weight loss, exercise or oral medications.

Clinical studies have clearly demonstrated the benefits of good blood glucose control. Good glycemic control can delay the development of microvascular and macrovascular complications. However, achieving good glucose metabolic control is not easy. It is especially challenging in infants and toddlers with type I diabetes. Several factors contribute to the difficulty in managing diabetes in these young children, such as unpredictable insulin pharmacokinetics, variable and unpredictable eating patterns and activity, increased sensitivity to small amounts of insulin, threat of hypoglycemia, and difficulty in managing hypoglycemic events. Said problems can lead to widely fluctuating blood glucose levels including frequent hypoglycemic episodes, which could, except from the immediate life threatening event, have adverse developmental effects in the long term.

During insulin biosynthesis, insulin hormone is synthesized as a single polypeptide, preproinsulin, which is processed into proinsulin and subsequently proteolytically processed into insulin inside insulin granules of beta cells. Proinsulin consists of two polypeptides, the so called A- and B-chains, connected by sulfide bridges and by a fragment called C-peptide. C-peptide serves as a linker between the A- and the B-chains of insulin and facilitates the efficient assembly, folding, and processing of insulin in the endoplasmic reticulum. Proinsulin maturates into active peptide insulin by releasing C-peptide and leaving 2 peptide chains, the B- and A-chains, linked by 2 disulfide bonds. Equimolar amounts of C-peptide and insulin are then stored in secretory granules of the pancreatic beta cells and both are eventually released to the portal circulation. Postprandial peak of glucose stimulates simultaneous release of equal-molecular quantity of insulin, C-peptide and certain quantity of proinsulin, which is not processed in beta-cells. Said products of insulin endogenous synthesis are released together in healthy people during hyperglycemia. Consequently, when insulin synthesis is impaired, patients will also become C-peptide as well as proinsulin deficient. Recent research indicates that these peptides may play an important physiological role in diabetes related complications. C-peptide helps to prevent neuropathy and other vascular deterioration related symptoms of diabetes mellitus. However, in the current clinical practice insulin treatment does not include co-administration of C-peptide and proinsulin.

The significance of mimicking the normal physiology function is further supported by the fact that pancreas transplantation, which restores not only insulin secretion, but also that of C-peptide and proinsulin release, is associated with prevention and even reversal of diabetic complications. Pancreas transplantation reduces diabetic lesions after ten years of normoglycemia, when compared to treatment with recombinant insulin. This study clearly indicates the importance of C-peptide and proinsulin for the normal metabolic state.

C-peptide has an important role in many of the diabetes related complications. Vascular complications, such as decreased blood flow in the extremities can be ameliorated by C-peptide. Several studies report a reduction of microvascular complications in patients with type I, as well as type II, diabetes with circulating concentrations of C-peptide close to physiological levels.

It has been discovered that human proinsulin is internalized into target tissues, e.g., fat cells. This finding supports the assumption that proinsulin plays an active role and is necessary for attainment of natural hormonal homeostasis. Additional studies demonstrate that insulin receptor binding is enhanced by the presence of human proinsulin. Accumulation evidence suggests that exogenous proinsulin may enhance insulin receptor binding, and improve its glucose lowering effect.

Under normal physiological condition, all three proteins (insulin, proinsulin and C-peptide) are being secreted from the pancreas into the portal system, while only a fraction of these proteins, which is not metabolized in the liver, reaches the peripheral circulation. In the current clinical practice, using parenteral administration of insulin, insulin gets initially into to the circulation, generating a high concentration peak in the blood, and only a fraction that was not consumed by the peripheral tissues (such as for example adipose and muscles tissues) is being metabolized in the liver.

Thus, it may be concluded that administration of proinsulin and C-peptide, in parallel to insulin administration, may preserve the normal physiology function and reduce the risk of diabetes complications. In order to mimic the normal physiology, the combination of insulin, proinsulin and C-peptide should be provided via the portal system (administered orally and subsequently absorbed through the gastrointestinal tract), as effected by pancreatic secretion, in order to enable first pass metabolism in the liver.

International Patent Application/Publication No. WO2009/087633, of the inventors of the present invention, discloses a pharmaceutical composition for oral use, comprising an oil having particulate matter suspended therein, wherein the particulate matter comprises: (a) a polysaccharide in intimate non-covalent association with silica nanoparticles having a hydrophobic surface, wherein the size of the silica nanoparticles is between 1-100 nanometers; and (b) a protein or peptide having therapeutic activity, non-covalently associated with said silica nanoparticles and the polysaccharide.

International Patent Application/Publication No. WO 2009/087634, of the inventors of the present invention, discloses a pharmaceutical composition for oral use comprising an oil having particulate matter suspended therein, wherein the particulate matter comprises (a) a polysaccharide in intimate non-covalent association with silica particles having a hydrophobic surface, wherein the size of the silica particles is between 1-100 nanometers; and (b) an insulin protein non-covalently associated with said silica particles and the polysaccharide.

International Patent Application/Publication No. WO2011/004376, of the inventors of the present invention, discloses a matrix carrier composition for use in pharmaceutical delivery system, the composition comprising an intermolecular association of at least: a first solid phase comprising nanoparticles having hydrophobic surface, wherein the size of the nanoparticles is in the range of about 5-1000 nm; a second solid phase, comprising a biopolymer having hydrophilic and hydrophobic parts; and a continuous phase comprising oil associated with said first and said second solid phases.

U.S. Pat. No. 4,654,324 to Chance et al. is directed to a pharmaceutical composition which comprises human proinsulin in association with a pharmaceutically acceptable carrier, wherein the composition is useful in controlling a diabetic condition and in promoting attainment of natural hormonal homeostasis, thereby preventing or substantially diminishing or retarding diabetic complications.

US Patent Application No. 2003/0220229 is directed to proinsulin peptide compounds that modulate an immunological response by T cells of Type I diabetic subjects, to pharmaceutical compositions comprising same, to the diagnostic assays for Type I diabetes using the proinsulin peptide compounds and to the methods for inhibiting the development or progression of Type I diabetes in a subject by administering a proinsulin peptide compound.

U.S. Pat. No. 7,964,558 encompasses a method of treating diabetes and/or microvascular diabetic complications comprising subcutaneously administering C-peptide or a pharmaceutical composition comprising C-peptide to a patient once daily.

The combined use of insulin, proinsulin and C-peptide has been suggested by Chance et al. in U.S. Pat. No. 4,652,547 and U.S. Pat. No. 4,652,548. Chance disclosed a pharmaceutical composition for parental administration comprising human insulin, human C-peptide, and human proinsulin, wherein the molar ratio of human insulin to human C-peptide, is from about 1:4 to about 4:1, and the weight ratio of human insulin to human proinsulin is from about 1:100 to about 100:1, and wherein the pharmaceutical composition is useful in treating diabetics and in promoting attainment of natural hormonal homeostasis, thereby preventing or substantially diminishing or retarding diabetic complications.

The use of proinsulin peptide for treating Type I diabetes is also disclosed in US 2003/0220229 to Griffin et al.

There remains an unmet need for orally-administrable pharmaceutical compositions comprising a combination of bioactive proteins including insulin, proinsulin and C-peptide, which would provide portal delivery of said bioactive proteins, thus providing natural processing route of endogenous insulin and allowing mimicking endogenous pancreatic physiologic function. In addition to being useful in diabetes treatment, such pharmaceutical compositions would allow reducing diabetes complications and risks.

### SUMMARY OF THE INVENTION

The present invention is directed to orally administrable pharmaceutical compositions comprising a combination of at least two proteins or peptides associated with insulin endogenous synthesis, selected from the group consisting of insulin, proinsulin and C-peptide, and a pharmaceutically acceptable carrier, suitable for oral administration that provides portal delivery of said bioactive proteins. The exemplary orally administrable compositions comprise a particulate non-covalently associated intimate mixture of pharmacologically inert silica particles having a hydrophobic surface, a polysaccharide, and at least two bioactive proteins selected from as the group consisting of insulin, proinsulin and C-peptide; said mixture being suspended or embedded in an oil or mixture of oils.

Pharmaceutical compositions of the present invention, comprising a combination of bioactive proteins associated with glucose metabolism, not only provide the preferred delivery route for insulin administration, but also allow imitation of the conditions of natural production of insulin. The present invention further provides therapeutic uses of said pharmaceutical compositions.

As disclosed herein for the first time, the orally administered composition comprising mixtures of insulin, proinsulin and C-Peptide provided normoglycemic control when administered together with reduced dosages of injected insulin. Accordingly, it is now disclosed that orally-administrable combinations of insulin, proinsulin and C-peptide or a mixture of at least two of these three agents, provides treatment of diseases related to glucose metabolic pathways. Additionally, said combinations administered in a suitable oral-delivery vehicle, allow reducing the dosage of injected insulin and decrease fluctuations in glucose concentration levels. Furthermore, in contrast to parenterally administered insulin, or a combination of said bioactive proteins, the oral administration of these combinations in a vehicle that enables absorption via portal delivery according to the principles of present invention, mimics the normal physiological path of pancreatic secretion, is safe and effective and as such overcomes the drawbacks of the prior art formulations. It is to be understood explicitly that the compositions of the invention may be orally administered in any composition that provides portal delivery of the protein ingredients in active form.

Specifically and unexpectedly, insulin, proinsulin and C-peptide within the oil-based compositions of the present invention were found to remain intact, active and unharmed when incubated in a highly acidic environment in the presence of the digestive protease pepsin. The pharmaceutically active ingredients are associated with the delivery vehicle components and with each other via non-covalent bonds, allowing release of each of the active ingredients, without any chemical modification that might interfere with the known activity of each of the bioactive proteins.

Therefore, according to one aspect, the invention provides a pharmaceutical composition for oral use comprising at least two bioactive proteins associated with glucose metabolism, selected from the group consisting of insulin, proinsulin and C-Peptide in a delivery vehicle, adapted for oral administration that provides portal protein delivery of bioactive proteins, the delivery vehicle comprising an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with the at least two bioactive proteins, and wherein the weight ratio of insulin to proinsulin is from about 25:1 to about 1:2, the weight ratio of insulin to C-Peptide is from about 3:1 to about 1:2 and the weight ratio of silica to the bioactive proteins is from about 100:1 to about 1:1.

According to some embodiments, the weight ratio of silica particles to insulin is within the range of 100:1 to 1:1. According to some embodiments, the weight ratio of silica particles to proinsulin is within the range of 200:1 to 2:1. According to some embodiments, the weight ratio of silica particles to C-peptide is within the range of 200:1 to 1:1.

According to some embodiments, the weight ratio of polysaccharide to insulin is within the range of 200:1 to 5:1. According to some embodiments, the weight ratio of polysaccharide to proinsulin is within the range of 400:1 to 5:1. According to some embodiments, the weight ratio of polysaccharide to C-peptide is within the range of 400:1 to 5:1.

According to some embodiments, each of the bioactive proteins is non-covalently associated with said polysaccharide and silica particles. According to particular embodiments, insulin is non-covalently associated with polysaccharide and silica particles. According to other particular embodiments, proinsulin is non-covalently associated with polysaccharide and silica particles. According to additional particular embodiments, C-Peptide is non-covalently associated with polysaccharide and silica particles.

According to some embodiments, at least two bioactive proteins are associated with each other via non-covalent bonds. According to specific embodiments, insulin is non-covalently associated with proinsulin and/or C-Peptide. According to yet further embodiments, proinsulin is non-covalently associated with C-Peptide.

According to some embodiments, one of the at least two bioactive proteins is insulin. According to further embodiments, the pharmaceutical composition comprises insulin, proinsulin and C-peptide. According to still further embodiments, the pharmaceutical composition comprises insulin, proinsulin and C-Peptide non-covalently associated with the polysaccharide and silica particles, wherein the mixture of the bioactive proteins, silica particles and polysaccharide is suspended in the oil matrix. According to yet further embodiments, the pharmaceutical composition comprises insulin and proinsulin non-covalently associated with the polysaccharide and silica particles, wherein the mixture of the bioactive proteins, silica particles and polysaccharide is suspended in the oil matrix. According to still further embodiments, the pharmaceutical composition comprises insulin and C-Peptide non-covalently associated with the polysaccharide and silica particles, wherein the mixture of the bioactive proteins, silica particles and polysaccharide is suspended in the oil matrix. In other embodiments, the pharmaceutical composition comprises proinsulin and C-Peptide non-covalently associated with the polysaccharide and silica particles, wherein the mixture of the bioactive proteins, silica particles and polysaccharide is suspended in the oil matrix. In further embodiments, each one of insulin, proinsulin and C-peptide is non-covalently associated with polysaccharide and silica particles. In other embodiments, the pharmaceutical composition comprises insulin, proinsulin and C-Peptide non-covalently associated with each other and with the polysaccharide and silica particles, wherein the mixture of the bioactive proteins, silica particles and polysaccharide is suspended in the oil matrix.

In some embodiments, the oil is a mixture of oils. According to various embodiments, the oil components of the composition are 1-75% of the total weight of the composition. According to some alternative embodiments at least 30%, at least 40%, at least 50%, at least 60% of the composition is oil. According to yet another embodiment, at least 65% of the composition is oil. Without wishing to be limited by theory or mechanism of action it is suggested that the oil components take part in formation of non-covalent binding of insulin, proinsulin and/or C-Peptide to silica particles.

According to some embodiments, the oil comprises an oil having a melting temperature of at least 5 to 10° C. According to further embodiments, said oil comprises a mixture of oils selected from natural vegetable oils and synthetic analogues thereof. Each possibility represents a separate embodiment of the invention.

According to yet another embodiment, the polysaccharide comprises a branched polysaccharide. According to yet another embodiment, said branched polysaccharide is selected from the group consisting of starch, starch derivates, amylopectin, and glycogen. Each possibility represents a separate embodiment of the present invention. According to yet another embodiment, said branched polysaccharide is a starch. According to yet another embodiment said branched polysaccharide has a melting temperature of not more than 400° C.

According to yet another embodiment, said pharmaceutical composition is anhydrous.

According to another embodiment, a size of said silica nanoparticles is within the range of 1 to 100 nanometers. According to yet another embodiment, the size of said silica nanoparticles is within the range of 5 to 30 nanometers. According to yet another embodiment said silica nanoparticles have a melting temperature of not less than 600° C. According to yet another embodiment, the hydrophobic surface of said silica particles comprises hydrocarbon moieties.

According to yet another embodiment, the pharmaceutical composition further comprises at least one additional biopolymer. According to some embodiments, the additional biopolymer may include a linear polysaccharide selected from the group consisting of soluble, poorly soluble or insoluble linear polysaccharide. Non limiting examples of such linear polysaccharides include: cellulose, chitin, amylose, glycosaminoglycans (GAG), mucopolysacchrides and glucans (e.g. alpha glucan, beta glucan). According to some embodiments, the additional biopolymer may be a cyclic oligosaccharide (also referred to as cyclodextrin). According to some currently preferred embodiments, the cyclodextrin is β-cyclodextrin. According to additional embodiments, the pharmaceutical composition of the invention may further include at least one of a saccharide and/or an oligosaccharide. Each possibility represents a separate embodiment of the present invention.

According to additional embodiments, the additional biopolymer may comprise a structural protein. According to some embodiments, said structural protein is selected from the group consisting of elastin, collagen, keratin and fibrinogen. Each possibility represents a separate embodiment of the present invention.

According to yet another embodiment said oil comprises a mixture of oils. According to yet another embodiment, said oil comprises a mixture of oils selected from natural vegetable oils and synthetic analogues thereof. Each possibility represents a separate embodiment of the present invention. According to yet another embodiment, said oil comprises an oil having a melting temperature of at least 5 to 10° C.

According to yet another embodiment, the weight of said particulate matter is no more than 80% of the weight of said pharmaceutical composition. According to various embodiments the weight of the particulate matter of the composition is 25-80% of the total weight of the composition. According to some alternative embodiments the weight of the particulate matter is no more than 70%, preferably not more than 60%, more preferably not more than 50%, even more preferably not more than 40% of the weight of the pharmaceutical composition. According to yet another embodiment, the weight of the particulate matter is at least 35% of the total weight of the composition.

According to further embodiments, the pharmaceutical compositions of the invention are formulated in a form selected from the group consisting of liquid, solid, semi-solid, gel and microencapsulated forms. Each possibility represents a separate embodiment of the invention. According to further embodiments, the pharmaceutical compositions are formulated in a dosage form selected from the group consisting of a capsule, microcapsule, tablet, microencapsulated tablet, powder, suspension, paste and a combination thereof. Each possibility represents a separate embodiment of the invention. In some embodiments, the dosage form is a tablet. The tablet can comprise a dry-coated tablet. In other embodiments, the dosage form is a microencapsulated tablet. The microencapsulated tablet may further comprise an excipient. In further embodiments, the excipient is added to the oil phase of the composition to obtain a plurality of droplets containing oil having particulate matter suspended therein. The excipient may be present in the composition in a weight percent ranging from about 20% to about 80% of the total weight of the composition. According to further embodiments, the excipient may include additional polysaccharide.

In another aspect, the present invention provides a pharmaceutical composition for oral use, comprising at least two bioactive proteins associated with glucose metabolism selected from the group consisting of an insulin protein, proinsulin and C-peptide, in a delivery vehicle, adapted for oral administration that provides portal delivery of bioactive proteins, wherein the weight ratio of insulin to proinsulin is from about 25:1 to about 1:2 and the weight ratio of insulin to C-Peptide is from about 3:1 to about 1:2. According to some embodiments, the delivery vehicle is selected from the group consisting of permeation enhancers, lipid delivery vehicles, liposomes, lipid nanoparticles, polymer matrices, polymeric microspheres, self-emulsifying drug delivery systems (SEDDS), molecules comprising alkoxy groups, non-ionic surfactants, nano-particle delivery systems, oil-based matrices and combinations thereof.

According to some embodiments, one of the at least two bioactive proteins is insulin. According to further embodiments, the pharmaceutical composition comprises insulin, proinsulin and C-peptide. According to other embodiments, the pharmaceutical composition comprises a combination of insulin and proinsulin. According to alternative embodiments, the pharmaceutical composition comprises a combination of insulin and C-peptide. In a certain embodiment, the pharmaceutical composition comprises a combination of proinsulin and C-peptide.

The pharmaceutical compositions according to the embodiments of the present invention may further comprise at least one additional component, selected from the group consisting of antioxidants, amino acids, polypeptides, absorption enhancers, non-insulin glucose lowering drugs, blood pressure lowering drugs and combinations thereof. Each possibility represents a separate embodiment of the present invention.

According to some embodiments, the pharmaceutical composition comprises at least one antioxidant. The antioxidant as referred to herein means a molecule that balances the endogeneous antioxidant defense system which may be impaired during diabetes or metabolic related disease. The antioxidant reduces diabetic and metabolic related complications by destroying free radicals or oxidants involved in oxidative stress and enhances insulin secretion and insulin sensitization. According to some embodiments, the at least one antioxidant is non-covalently associated with the silica particles and/or the polysaccharide. According to some embodiments, the antioxidant is superoxide dismutase (SOD). According to some embodiments, the antioxidant is glutathione peroxidase. According to some embodiments, the antioxidant is a vitamin. According to various embodiments, the vitamin may be selected from vitamin A, vitamin C, vitamin E or any combination thereof. Additional antioxidants that may be included in the pharmaceutical composition according to some embodiments of the invention include: glutathione, α-lipoic acid, cartenoids, polyphenols, coenzyme Q₁₀, antioxidant minerals (e.g. copper, zinc, manganese, chrome and selenium) and cofactors (e.g. folic acid, vitamins B₁, B₂, B₆ and B₁₂).

According to some embodiments, the pharmaceutical composition comprises at least one free amino acid. According to some embodiments the free amino acid is selected from the group consisting of arginine, leucine, isoleucine, aspartic acid, glutamic acid, glutamine, asparagine, histidine, phenylalanine and any combination and derivatives thereof. Each possibility represents a separate embodiment of the invention. Without wishing to be limited by theory or mechanism of action it is suggested that insulin response may be increased by the co-ingestion of a free amino acid.

According to some embodiments, the pharmaceutical composition comprises at least one absorption enhancer. According to further embodiments, the absorption enhancer is selected from a medium chain fatty acid, a polyol or a combination thereof. Each possibility represents a separate embodiment of the invention.

According to some embodiments, the pharmaceutical composition comprises at least one glucose metabolism related treatment agent. According to further embodiments, the glucose metabolism related treatment agent is any glucose metabolism related treatment agent known in the field.

According to further embodiments, the pharmaceutical composition is formulated in a form selected from the group consisting of liquid, solid, semi-solid, gel and microencapsulated forms. Each possibility represents a separate embodiment of the invention. According to further embodiments, the pharmaceutical composition is formulated in a dosage form selected from the group consisting of a capsule, microcapsule, tablet, microencapsulated tablet, powder, suspension, paste and a combination thereof. Each possibility represents a separate embodiment of the invention.

According to some embodiments, the pharmaceutical compositions of the invention are for treating of a disease related to glucose metabolic pathways in a subject. According to some embodiments, the pharmaceutical compositions are for use in treating diabetes in a subject. According to various embodiments, the diabetes is type I diabetes (also referred to as juvenile diabetes or an insulin-dependent diabetes). According to additional embodiments, the diabetes is type II diabetes (also referred to as a non-insulin-dependent diabetes, diabetes related to obesity, adolescent diabetes or adult diabetes). According to additional embodiments, the diabetes is gestational diabetes. According to some embodiments, the pharmaceutical compositions are for treating obesity in a subject.

According to some embodiments, the pharmaceutical compositions are for use in treating diabetes in combination with parenterally administered insulin. According to additional embodiments, the pharmaceutical compositions are for use in combination with lower therapeutic doses of parenterally administered insulin, compared to the dose required without said pharmaceutical composition. According to further embodiments, the pharmaceutical compositions are for use in combination with lower therapeutic doses of parenterally administered insulin, compared to the dose required with orally administered insulin in a suitable delivery vehicle.

According to further embodiments, the compositions of the present invention are for use in the treatment of metabolic disease or condition. The metabolic disease or condition may be selected from the group consisting of metabolic syndrome, hyperlipidemia, hypercholesterolemia, hypertriglyceridemia, hyperglycemia, insulin resistance, hepatic steatosis, kidney disease, fatty liver disease, non-alcoholic steatohepatitis and a combination thereof.

In further embodiments, the pharmaceutical compositions are for use in treating or preventing a diabetes-related complication in a subject. The diabetes-related complication may be selected from the group consisting of decreased blood flow in the extremities, retinopathy, cardiovascular disorder, peripheral artery disorder, lower limb gangrenous inflammation and a combination thereof.

In some embodiments, the pharmaceutical compositions are for use in treating a disease, a condition or a complication selected from the group consisting of diabetes, metabolic disease or condition, diabetes-related complication and a combination thereof. In alternative embodiments, the invention provides use of the pharmaceutical compositions for treating disease, a condition or a complication selected from the group consisting of diabetes, metabolic disease or condition, diabetes-related complication and a combination thereof. Each possibility represents a separate embodiment of the invention.

According to yet another embodiment, the present invention provides a method for treating diabetes in a subject in need thereof, comprising orally administering to said subject the pharmaceutical composition of the invention. According to yet another embodiment, the present invention provides use of the pharmaceutical compositions of the invention for treating diabetes in a subject. According to yet another embodiment, the present invention provides a method for treating one or more diabetes-related complications in a subject in need thereof, comprising orally administering to said subject the pharmaceutical composition of the invention. According to yet another embodiment, the present invention provides use of the pharmaceutical compositions of the invention for treating one or more diabetes-related complications in a subject in need thereof.

According to some embodiments, said diabetes is selected from the group consisting of: Type I diabetes, Type II diabetes and gestational diabetes. Each possibility represents a separate embodiment of the present invention.

According to further embodiments, the present invention provides a method for treating a metabolic disease or condition in a subject in need thereof, comprising orally administering to said subject the pharmaceutical composition of the invention. According to additional embodiment, the present invention provides a method for treating obesity and/or obesity-related conditions in a subject in need thereof, comprising orally administering to said subject the pharmaceutical composition of the invention. According to yet additional embodiments, the present invention provides a method for treating a metabolic disease or condition other than diabetes or obesity in a subject in need thereof, comprising orally administering to said subject the pharmaceutical composition of the invention. According to some embodiments, the metabolic disease or condition is selected from the group consisting of metabolic syndrome, hyperlipidemia, hypercholesterolemia, hypertriglyceridemia, hyperglycemia, insulin resistance, hepatic steatosis, fatty liver disease and non-alcoholic steatohepatitis. Each possibility represents a separate embodiment of the invention. According to yet another embodiment, the present invention provides use of the pharmaceutical compositions of the invention for treating a metabolic disease or condition in a subject in need thereof.

According to further embodiments, the method of treating diabetes comprises administering the pharmaceutical composition of the present invention instead of parenterally administered insulin. According to other embodiments, the method comprises administering the pharmaceutical composition in combination with parenterally administered insulin.

According to still further embodiments, the pharmaceutical composition is administered in combination with lower therapeutic doses of injected insulin, compared to the dose required without orally administered combination of the at least two molecules associated with glucose metabolism in a suitable delivery vehicle. According to yet further embodiments, the pharmaceutical composition is administered in combination with lower therapeutic doses of injected insulin, compared to the dose required with orally administered insulin in a suitable delivery vehicle.

According to some embodiments, the subject is a human. According to yet another embodiment, the subject is a non-human mammal According to yet another embodiment, said subject is pregnant.

Other objects, features and advantages of the present invention will become clear from the following description.

### BRIEF DESCRIPTION OF THE DRAWINGS

FIG. 1 shows a chromatogram of formulation A obtained by HPLC.

FIG. 2 shows a HPLC chromatogram of formulation A after a 24 hours dissolution testing, indicating that the majority of the API remained intact.

FIG. 3 shows mean glucose concentration (GC) for Oshadi ICP (Oshadi GC, grey solid line) and insulin adjusted placebo GC (aGC, black solid line) over daytime (7:00-24:00) at the third administration day. The black dotted line indicates GC of 180 mg/dL.

FIG. 4 shows mean daytime AUC GC>180 mg/dL.

### DETAILED DESCRIPTION OF THE INVENTION

The present invention encompasses pharmaceutical compositions comprising a combination of at least two agents, selected from proteins and peptides associated with insulin endogenous synthesis, including insulin, proinsulin and C-Peptide, also referred herein as pharmaceutically active ingredients, in a delivery vehicle suitable for oral administration of protein drugs, adapted to provide portal delivery of said proteins.

### Insulin, Proinsulin and C-Peptide

Natural insulin is derived from a preproinsulin protein which is secreted in the body with A-chain, C-peptide, a B-chain, and a signal sequence. Initially, the signal sequence is removed leaving the remaining A-chain, C-peptide and a B-chain, also termed “proinsulin”. After the C-Peptide is cut off, the A-chain and B-chain are left to form insulin.

The terms “insulin protein” and “insulin” as used herein include rapid-acting insulin, very rapid-acting insulin, intermediate-acting insulin, and long-acting insulin. Non-limiting examples of rapid-acting insulin are lyspro insulin (Lysine-Proline insulin, sold by Eli Lilly as Humalog™), glu-lysine insulin (sold by Sanofi-Aventis as Apidra™), Actrapid™ and NovoRapid™ (both available from Novo Nordisk), aspart insulin (sold by Novo Nordisk as Novolog™). A non-limiting example of very rapid-acting insulin is Viaject™, marketed by Biodel. Non-limiting examples of intermediate-acting insulin are NPH (Neutral Protamine Hagedorn) and Lente insulin. A non-limiting example of long-acting insulin is Lantus™ (insulin glargine). In some preferred embodiments, the insulin is Insugen™ from Biocon™ Insulin also includes a mixture of different types of insulin. Some non-limiting examples of a such a mixture are Mixtard® 30, Mixtard® 40, and Mixtard® 50, which are mixtures of different proportions of short-acting insulin and NPH (intermediate duration) insulin. The insulin may be selected from a naturally occurring insulin and a modified form of insulin. It will be clear from the present disclosure that methods and compositions of the present invention are suitable for every type of natural and modified insulin known in the art.

The ratio insulin:proinsulin and insulin:C-Peptide in the pharmaceutical compositions of the invention may vary depending on the types of diabetes which is treated.

Pancreas of a healthy individual release from about 5-7% to about 30% proinsulin relatively to the molecular concentration of released insulin and about 52% C-peptide—amounts corresponding to equal molecular concentrations of insulin and C-peptide and from 2.7 to 18.6% of proinsulin. Thus, according to some embodiments, the weight ratio of insulin to proinsulin varies from 25:1 to 1:2. Alternatively, the weight ratio of insulin to proinsulin varies from 2:1 to 1:2. Alternatively, the weight ratio of insulin to proinsulin varies from 2:1 to 1:1.5. Alternatively, the weight ratio of insulin to proinsulin varies from 2:1 to 1:1. According to some embodiments, the weight ratio insulin to C-peptide varies from 3:1 to 1:2. Alternatively, the weight ratio of insulin to C-peptide varies from 3:1 to 1:1.5. Alternatively, the weight ratio of insulin to proinsulin varies from 2:1 to 1:1.5. Alternatively, the weight ratio of insulin to proinsulin varies from 2:1 to 1:1.

According to further embodiments, the weight ratio of proinsulin to C-peptide is from about 1:10 to about 2:1. For different types of diabetes or metabolic diseases different ratio of C-Peptide: insulin and proinsulin: insulin may be used. For different types of diabetes or metabolic diseases different ratio of proinsulin:C-Peptide may be used.

Without wishing to be bound by any theory or mechanism, treating diabetes with a combination of insulin, proinsulin and/or C-Peptide, is advantageous compared to treatment with insulin alone, due to the specific metabolism of these compounds. In a healthy individual about 5% of endogenous insulin relates to the regulation of blood glucose level.

Most of the insulin is used for other homeostasis pathways, such as, amino acid and neurotransmitters metabolism, among others. Most of the insulin remains in the liver and participates in various metabolic pathways that affect the nervous system and the whole body. The liver utilizes about 75-85% of the insulin which is secreted by the pancreases, while 90-95% of proinsulin and about 100% of C-Peptide pass the liver almost without delays. Moreover, experimental data show that the difference in insulin peak concentration between portal vein and systemic circulation is in the order of about 4-5. It is also known that in the pancreas and in the portal vein insulin is in a dynamic equilibrium between insulin monomer, insulin dimer, insulin tetramer, insulin hexamer and proinsulin with C-peptide. Accordingly, the formulations of the invention include the natural combination of active pharmaceutical agents: insulin, C-Peptide and/or proinsulin thereby enabling a wide range of physiological activities which imitate the pancreatic function in different metabolic pathways including regulation of blood glucose levels.

According to some embodiments, the pharmaceutical composition comprises a combination of insulin and proinsulin. According to other embodiments, the pharmaceutical composition comprises a combination of insulin and C-Peptide. According to other additional embodiments, the pharmaceutical composition comprises a combination of proinsulin and C-Peptide. According to the exemplified embodiments, the pharmaceutical composition comprises a combination of insulin, proinsulin and C-Peptide.

According to some embodiments, the pharmaceutically active ingredients are associated via non-covalent bonds. According to further embodiments, insulin is non-covalently associated with proinsulin and/or C-Peptide. According to still further embodiments, proinsulin is non-covalently associated with C-Peptide. Without wishing to being bound by any specific theory or mechanism of action, the non-covalent association between the bioactive proteins ensures release of each of the active ingredients, without any chemical modification that might interfere with the known activity of each of the bioactive proteins. According to further embodiments, the non-covalently bound bioactive proteins remain intact upon release thereof from the delivery vehicle.

### Additional Components

The pharmaceutical compositions comprising the mixture of at two of insulin, proinsulin and C-Peptide can further comprise additional components, selected from the group consisting of antioxidants, amino acids, polypeptides, insulin enhancers, absorption enhancers, non-insulin glucose lowering drugs, blood pressure lowering drugs and combinations thereof.

According to some embodiments, the pharmaceutical composition comprises at least one antioxidant. The antioxidant as referred to herein means a molecule that balances the endogeneous antioxidant defense system which may be impaired during diabetes. The antioxidant reduces diabetic complications by destroying free radicals or oxidants involved in oxidative stress and enhances insulin secretion and insulin sensitization. According to some embodiments, the antioxidant is superoxide dismutase (SOD). According to some embodiments, the antioxidant is glutathione peroxidase. According to some embodiments, the antioxidant is a vitamin. According to various embodiments, the vitamin may be selected from vitamin A, vitamin C, vitamin E or any combination thereof. Additional antioxidants that may be included in the pharmaceutical composition according to some embodiments of the invention include: glutathione, α-lipoic acid, omega-3, cartenoids, bioflavonoids, polyphenols, coenzyme Q₁₀, antioxidant minerals (e.g. copper, zinc, manganese, chrome and selenium) and cofactors (e.g. folic acid, vitamins B₁, B₂, B₆ and B₁₂).

Antioxidant minerals may include organic salts of zinc, organic salts of chrome, organic salts of copper, organic salts of manganese and seleno-amino acids Non limiting examples of organic salts of zinc include zinc acetate, zinc butyrate, zinc citrate, zinc gluconate, zinc glycerate, zinc glycolate, zinc formate, zinc lactate, zinc picolinate, zinc proprionate, zinc tartrate and zinc undecylenate. Non limiting examples of organic salts of chrome include chromium citrate, chromium acetate, chromium propionate and chromium malonate. According to some embodiments the seleno-amino acid is selected from the group consisting of selenocysteine and selenomethionine.

According to some embodiments, the pharmaceutical composition comprises at least one free amino acid. According to some embodiments the free amino acid is selected from the group consisting of arginine, leucine, isoleucine, aspartic acid, glutamic acid, glutamine, asparagine, histidine, phenylalanine and any combination and derivatives thereof. Each possibility represents a separate embodiment of the invention. Without wishing to being limited by theory or mechanism of action it is suggested that insulin response may be increased by the co-ingestion of a free amino acid.

According to some embodiments, the composition comprises one or more enhancers, such as, an enhancer of the insulin protein. Non limiting examples of insulin enhancers include: dodecylmaltoside, octylglucoside, and dioctyl sodium sulphosuccinate. The enhancer may be a cofactor of the insulin protein. Non limiting example of an insulin cofactor is chromium.

According to some embodiments, the composition comprises one or more glucose metabolism associated treatment agent.

According to some embodiments, the composition comprises one or more additional polypeptides, such as, but not limited to, calcitonin, glucagon-like peptide (GLP), glucagon-like peptide analog, leptin or amylin. Each possibility represents a separate embodiment of the invention. Glucagon-like peptides and their analogues are well known in the art, and are described, inter alia, in Eleftheriadou I. et al. (The effects of medications used for the management of diabetes and obesity on postprandial lipid metabolism. Curr. Diabetes Rev 4(4):340-56, 2008 and Vaidya H B et al., Glucagon like peptides-1 modulators as newer target for diabetes and metabolic related disorders. Curr. Drug Targets 9(10):911-20, 2008). Each possibility represents a separate embodiment of the present invention.

According to some embodiments, the composition comprises a non-insulin treatment agent related to carbohydrates metabolic pathways. The non-limiting examples of such drugs include gliclazade, sulfonylurea, metformin, rosiglitazone and glimepiride. Each possibility represents a separate embodiment of the invention. According to further embodiments, the pharmaceutical composition comprises blood pressure lowering drugs, such as, but not limited to angiotensin converting enzyme inhibitors, angiotensin receptor blockers, calcium channels blockers, beta blockers, rennin antagonist, aldosteron blockers and diuretics.

Typically, the choice of an additional component in the compositions and methods of the invention, depends on the required treatment. For example, for treating gestational diabetes (pregnancy diabetes), which is accompanied by lipid perioxidation, the following antioxidants would be considered: glutathione, glutathione peroxidase and vitamins, including, folic acid, vitamin E and a seleno-amino acid.

For treating Type II diabetes related to obesity, which is accompanied with excess activity of cytokines and kidney oxidative stress, one or more of the following antioxidants could be added: organic salts of Zn, omega-3 and SOD. Additionally, at least one free amino acid and/or biotin may be added to composition for treating Type II diabetes related to obesity.

For the treatment of Type I diabetes which is accompanied by amino acids misbalance, it would be useful to add one or more of the following: amino acids, antioxidants such as: vitamin K and/or organic salts of Zn, organic salts of chrome, a seleno-amino acid and cofactors such as vitamins of group B (to help nervous system and neurotransmitters formation), including, but not limited to, any one or more of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), Vitamin B7 (biotin), Vitamin B9 (folic acid), Vitamin B12 (various cobalamins), vitamin B complex and combinations thereof. Each possibility represents a separate embodiment of the present invention.

For the treatment of metabolic disorders, pectin and/or amylin can be added to the compositions comprising insulin, proinsulin and/or C-Peptide.

### Delivery Vehicles

The oral delivery of the active ingredients is afforded by means of a suitable protein delivery vehicle that achieves portal vein delivery of the combination of bioactive proteins. As used herein, the term “portal delivery” refers to the route of oral administration of a substance, which is followed by subsequent absorption of the administered substance through the gastrointestinal tract. A suitable delivery vehicle will provide release of the pharmaceutically active ingredients and absorption thereof from the gastrointestinal tract so as to maintain effective levels thereof in the bloodstream over the desired period of time to provide the desired therapeutic effect. According to the exemplary embodiments, the delivery vehicle comprises an oil-based matrix, comprising solid dry particulate matter suspended therein, wherein the particulate matter comprises the active ingredients. According to additional embodiments, the at least two bioactive proteins selected from the group consisting of insulin, proinsulin and C-Peptide constitute a part of the particulate matter of said delivery vehicle.

According to other embodiments, the delivery vehicle is any pharmaceutically acceptable carrier suitable for oral administration of proteins enabling portal delivery thereof, known in art. The non-limiting examples of said delivery vehicles include permeation enhancers, lipid delivery vehicles, liposomes, polymer matrices, polymeric microspheres, self-emulsifying drug delivery systems (SEDDS), molecules comprising alkoxy groups, non-ionic surfactants, nano-particle delivery systems and combinations thereof. Permeation enhancers may comprise, inter alia, surfactants, preferably anionic surfactants, alkylmaltosides, medium-chain fatty acids and salts thereof, such as, but not limited to, capric acid, caprates, caprylic acid and caprylate. Molecules comprising alkoxy groups, suitable for oral protein delivery include, for example, glycerol or polyethylene glycol. Certain non-limiting examples of proprietary vehicle formulations adapted for oral delivery of proteins include GIPET® and ELIGEN®. Examples of delivery vehicles for oral insulin include Chiasma's Transient Permeability Enhancer (TPE).

### Oil-Based Matrix Composition Comprising Suspended Particulate Matter.

According to some embodiments, the oral delivery vehicle comprises an intimate mixture of solid dry particulate ingredients in an oil-based matrix. In these embodiments, the matrix carrier compositions, also termed “pharmaceutical compositions”, comprise a particulate non-covalently associated mixture of pharmacologically inert silica nanoparticles having a hydrophobic surface, a polysaccharide and at least two bioactive proteins, selected from the group consisting of insulin, proinsulin and C-peptide, suspended, embedded or dispersed in an oil or mixture of oils.

According to some embodiments, the at least two bioactive proteins selected from the group consisting of insulin, proinsulin and C-Peptide are incorporated within a delivery vehicle comprising an oil having particulate matter suspended therein, wherein the particulate matter comprises a polysaccharide in intimate non-covalent association with silica nanoparticles having a hydrophobic surface. Accordingly, the pharmaceutical compositions of some embodiments of the present invention comprise at least two bioactive proteins associated with glucose metabolism, selected from the group consisting of insulin, proinsulin and C-Peptide in a delivery vehicle, adapted for oral administration that provides portal protein delivery of bioactive proteins, the delivery vehicle comprising an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with the at least two bioactive proteins, and wherein .the weight ratio of insulin to proinsulin is from about 25:1 to about 1:2, the weight ratio of insulin to C-Peptide is from about 3:1 to about 1:2 and the weight ratio of silica to the bioactive proteins is from about 100:1 to about 1:1. According to the exemplary embodiments, the present invention provides a pharmaceutical composition for oral use comprising insulin, proinsulin and C-Peptide in a delivery vehicle, adapted for oral administration that provides portal protein delivery of bioactive proteins, the delivery vehicle comprising an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with insulin, proinsulin and C-peptide, and wherein .the weight ratio of insulin to proinsulin is from about 25:1 to about 1:2, the weight ratio of insulin to C-Peptide is from about 3:1 to about 1:2 and the weight ratio of silica to insulin, proinsulin and C-Peptide is from about 100:1 to about 1:1.

The matrix formed by the silica nanoparticles, polysaccharide, and a combination of insulin, C-peptide and/or proinsulin is suspended, embedded or dispersed in oil. According to some embodiments, the insulin, C-peptide and/or proinsulin are non-covalently attached to the hydrophobic surfaces of the silica nanoparticles and to the hydrophilic and hydrophobic portions, regions or patches of the surface of the polysaccharide. According to some embodiments, the hydrophobic portion of the insulin, proinsulin and C-peptide is attached to the hydrophobic surfaces of the silica nanoparticles and the polysaccharide via non-covalent forces. According to some embodiments, the hydrophobic portion of the insulin, proinsulin and C-peptide is attached to the hydrophobic surfaces of the silica nanoparticles and the polysaccharide via non-covalent bonds. According to some embodiments, the hydrophilic portion of the insulin protein is also non-covalently attached to hydrophilic portion of the polysaccharide.

According to some embodiments, the pharmaceutical composition of the present invention is held together by non-covalent forces. According to some embodiments, the pharmaceutical composition of the present invention is held together by non-covalent bonds.

Without wishing to be bound by any theory or mechanism of action, the non-covalent forces between the components of the matrix composition enable the matrix composition to self-assemble when the components are mixed together, as described herein. In addition, or alternatively, the non-covalent forces cause the silica nanoparticles, polysaccharide, insulin, proinsulin and/or C-peptide to form an intimate mixture, and, optionally, to form a matrix which exhibits an ordered structure. Furthermore, the structure, otherwise referred to as a complex of the silica nanoparticle, polysaccharide, insulin, proinsulin and/or C-Peptide is dispersed, embedded or suspended within the oil phase of the matrix composition. As provided herein, the present invention provides compositions wherein the silica nanoparticles, polysaccharide, insulin, proinsulin and/or C-Peptide form a matrix that is impregnated and completely surrounded by the oil phase. Each possibility represents a separate embodiment of the present invention.

Without wishing to being bound by any specific theory or mechanism of action, the non-covalent association between the bioactive proteins and the particulate matter of the delivery vehicle allows release of each of the bioactive proteins from the delivery vehicle to the hepatic portal vein. In further embodiments, the bioactive proteins are released without any chemical modification that might interfere with the known activity of each of the bioactive proteins. According to further embodiments, the non-covalently bound bioactive proteins remain intact upon release thereof from the delivery vehicle.

The pharmaceutical composition of the invention comprises a structured, self ordered complex with hierarchy of structure and binding energies. This unique hierarchic structure is crucial for the biological activity and bioavailability of the combination of bioactive proteins contained within the ordered complex. According to one embodiment, the ordered structure provides protection of the basic units from disintegration and dissolution in the gastro-intestinal track and enables their transport through the mucosa as a whole.

Without wishing to being bound by theory or mechanism of action the hierarchies of the structures and binding energies of the compositions of the present invention promote the formation of tiny (20-200 nm) oil drops in which the particulate matter is suspended. These tiny oil drops maintain their internal structure and protect the pharmaceutically active ingredients from disintegrating and dissolving in the small intestine. According to some embodiments, the bioactive proteins associated with glucose metabolism such as, insulin, proinsulun and C-Peptide, remain intact, active and unharmed in the presence of the digestive protease pepsin. According to some embodiments, the pharmaceutical composition is stable in the presence of the digestive protease pepsin.

According to some embodiments, the weight ratio of silica particles to insulin is within the range of 100:1 to 1:1. According to further embodiments, the weight ratio of silica particles to insulin is within the range of 75:1 to 25:1. According to other embodiments, the weight ratio of silica particles to insulin is within the range of 20:1 to 3:1. According to some embodiments, the weight ratio of silica particles to proinsulin is within the range of 200:1 to 2:1. According to further embodiments, the weight ratio of silica particles to proinsulin is within the range of 150:1 to 50:1. According to yet further embodiments, the weight ratio of silica particles to proinsulin is within the range of 30:1 to 6:1.

According to some embodiments, the weight ratio of silica particles to C-peptide is within the range of 200:1 to 1:1. According to other embodiments, the weight ratio of silica particles to C-peptide is within the range of 200:1 to 2:1. According to further embodiments, the weight ratio of silica particles to C-peptide is within the range of 150:1 to 50:1. According to yet further embodiments, the weight ratio of silica particles to C-peptide is within the range of 40:1 to 6:1.

According to some embodiments, the weight ratio of polysaccharide to insulin is within the range of 200:1 to 5:1. According to further embodiments, the weight ratio of polysaccharide to insulin is within the range of 150:1 to 50:1. According to yet further embodiments, the weight ratio of polysaccharide to insulin is within the range of 30:1 to 7:1.

According to some embodiments, the weight ratio of polysaccharide to proinsulin is within the range of 400:1 to 5:1. According to further embodiments, the weight ratio of polysaccharide to proinsulin is within the range of 200:1 to 50:1. According to other embodiments, the weight ratio of polysaccharide to proinsulin is within the range of 50:1 to 5:1.

According to some embodiments, the weight ratio of polysaccharide to C-peptide is within the range of 400:1 to 5:1. According to further embodiments, the weight ratio of polysaccharide to C-peptide is within the range of 200:1 to 50:1. According to other embodiments, the weight ratio of polysaccharide to C-peptide is within the range of 60:1 to 12:1.

The term “oil having particulate matter suspended therein”, as used herein, refers to particulate matter that is in contact with oil. The composition as a whole need not be homogeneous with regard to the distribution of the particulate matter. Rather, the particulate matter is capable of being dispersed or suspended in the oil when agitated. The particulate matter need not be completely homogeneous, but rather is characterized by its composition containing the ingredients specified herein and its intimate contact with the oil of the present invention. Compositions wherein the particulate matter is agglomerated fall within the scope of the present invention.

According to yet another embodiment, the pharmaceutical composition comprising the particulate matter embedded in oil, further comprises at least one additional biopolymer. According to some embodiments, the additional biopolymer may include a linear polysaccharide selected from the group consisting of soluble, poorly soluble or insoluble linear polysaccharide. Non limiting examples of such linear polysaccharides include: cellulose, chitin, amylose, glycosaminoglycans (GAG), mucopolysacchrides and glucans (e.g. alpha glucan, beta glucan). According to some embodiments, the additional biopolymer may be a cyclic oligosaccharide (also referred to as cyclodextrin). According to some currently preferred embodiments, the cyclodextrin is β-cyclodextrin. According to additional embodiments, the pharmaceutical composition of the invention may further include at least one of a saccharide and/or an oligosaccharide. Each possibility represents a separate embodiment of the present invention.

According to additional embodiments, the additional biopolymer may comprise a structural protein. According to some embodiments, said structural protein is selected from the group consisting of elastin, collagen, keratin and fibrinogen. Each possibility represents a separate embodiment of the present invention.

According to further embodiments, the additional biopolymer is a dietary fiber, an insoluble fiber, a linear insoluble dietary fiber, a soluble dietary fiber or a linear soluble dietary fiber.

The terms “fiber” and “dietary fiber” as used herein includes unavailable carbohydrates, indigestible residue, and plant cell polysaccharides and lignin, all of which are resistant to hydrolysis by human digestive enzymes. The fibers may be members of the group: guar gum, pectin, fructo-oligosaccharides and derivatives thereof. Small amounts of other indigestible compounds, such as phytates, tannins, saponins and cutin, may be included in dietary fiber since these compounds are indigestible and associated with dietary fiber polysaccharides.

According to some embodiments, the composition of the present invention comprises a branched biopolymer, a linear polysaccharide, and an insoluble fiber. According to other embodiments, a composition of the present invention comprises a branched biopolymer, a polypeptide, and an insoluble fiber. An example of such is a composition comprising amylopectin, a branched polysaccharide; keratin, a polypeptide; and cellulose, an insoluble fiber. Other branched polysaccharides, polypeptides, and insoluble fibers disclosed herein are suitable as well. According to further embodiments, a composition of the present invention comprises a branched polysaccharide, a linear polysaccharide, and an insoluble fiber. An example of such is a composition comprising amylopectin, a branched polysaccharide; chitin, a linear polysaccharide; and cellulose, an insoluble fiber. Other branched and linear polysaccharides and insoluble fibers disclosed herein are suitable as well. Each possibility represents a separate embodiment of the present invention.

According to some embodiments, the weight of polysaccharides is greater than the weight of the silica. According to further embodiments, the weight of the polysaccharides is at least twice that of the silica, or 5 fold that of the silica or at least 10 times greater than the weight of silica particles. Each possibility represents a separate embodiment of the present invention.

According to some embodiments, the pharmaceutical composition of the present invention further comprises an additional oil component. The term “additional oil component” encompasses additional oil or a mixture of oils, as described elsewhere herein. According to some embodiments, the additional oil component comprises an antioxidant.

It is to be understood that said pharmaceutical composition is an oil based suspension that is devoid of an aqueous phase. According to some embodiments, the pharmaceutical composition is substantially free of water.

“Substantially free of water” as used herein refers, in one embodiment, to a component containing less than 2% water by weight. In another embodiment, the term refers to a component containing less than 1% water by weight. In another embodiment, the term refers to a component containing less than 0.8% water by weight. In another embodiment, the term refers to a component containing less than 0.6% water by weight. In another embodiment, the term refers to a component containing less than 0.4% water by weight. In another embodiment, the term refers to a component containing less than 0.2% water by weight. In another embodiment, the term refers to the absence of amounts of water that affect the stability of the pharmaceutically active agents in the composition. In another embodiment, the term refers to a composition manufactured without the use of any aqueous solvents.

### Silica Particles

According to some embodiments, the silica particles of compositions of the present invention are pharmacologically and biologically inert. According to some embodiments, the silica particles are composed of materials that are generally recognized as safe (GRAS). According to some embodiments, the silica particles are non-toxic. According to some embodiments, the silica particles are non-teratogenic. Each possibility represents a separate embodiment of the present invention.

Reference to silica (e.g. silicon dioxide, silicate or a combination thereof) nanoparticles of the present invention as having a “hydrophobic” surface encompasses silica particles having a surface that was modified to be hydrophobic. According to some embodiments, the silica particles are modified by chemically coating the surface with a hydrocarbon, thereby causing the silica nanoparticles to display hydrocarbon moieties on their surface. According to some embodiments, the coating causes the silica particles to display hydrocarbon moieties on their surface. The hydrocarbon moieties displayed on the nanoparticles surface may be selected from the group consisting of methyl, ethyl, propyl, isopropyl, butyl, isobutyl, T-butyl, pentyl, and iso-pentyl.

The term “hydrophobic” with reference to silica particles of the present invention refers to silica particles having a “hydrophobic” surface, wherein at least 40% of the silica nanoparticle surface is hydrophobic, at least 50% of the surface is hydrophobic, at least 60% of the surface is hydrophobic, at least 70% of the surface is hydrophobic, at least 80% of the surface is hydrophobic, at least 90% of the surface is hydrophobic, or at least 95% of the surface is hydrophobic. Optionally, 40-100% of the surface is hydrophobic, 50-100% of the surface is hydrophobic, 60-100% of the surface is hydrophobic, 70-100% of the surface is hydrophobic, 80-100% of the surface is hydrophobic, 90-100% of the surface is hydrophobic, 95-100% of the surface is hydrophobic, 40-60% of the surface is hydrophobic, 40-50% of the surface is hydrophobic, 40-70% of the surface is hydrophobic, or 40-80% of the surface is hydrophobic. Each possibility represents a separate embodiment of the present invention.

According to some embodiments, nanoparticles of the present invention are practically insoluble in water. The term “Practically insoluble” refers to a substance having a solubility of less than 100 parts per million weight/weight (ppm), less than 200 ppm, less than 80 ppm, less than 60 ppm, less than 50 ppm, less than 40 ppm, less than 30 ppm, less than 20 ppm, less than 15 ppm, or less than 10 ppm. Each possibility represents a separate embodiment of the present invention.

According to some embodiments, the silica particles are between about 1-100 nanometers (nm) in diameter. According to some embodiments, the diameter of the silica particles of the present invention is between 5-30 nm inclusive, between 2-100 nm inclusive, between 3-80 nm inclusive, between 4-70 nm inclusive, between 4-60 nm inclusive, 5-50 nm inclusive, between 5-40 nm inclusive, or having a mean diameter of between 6-25 nm inclusive.

According to some embodiments, the melting temperature of the silica particles of the exemplary compositions of the present invention fall within a range of melting temperatures particularly suitable for said compositions, such as, a melting temperature (Tₘ) of over 600° C., or Tₘ between 600-4500° C. Each possibility represents a separate embodiment of the present invention.

The impartment of a hydrophobic surface to the nanoparticles of the invention may be done by any method known in the art for imparting a hydrophobic surface to nanoparticles. A non-limiting example of such process includes the chemical modification of the surface of fumed silica, generating a decrease in the number of silanol groups. Silanol groups may be substituted with hydrophobic groups to obtain a hydrophobic silica. The hydrophobic groups may be: trimethylsiloxy groups, which are commonly obtained by treatment of fumed silica in the presence of hexamethyldisilazane. Silica compounds treated this way are known as “silica silylate”, and are commercially available under the names “Aerosil R812®” (Degussa) and “CAB-OSIL TS-530®” (Cabot). Dimethylsilyloxy or polydimethylsiloxane groups, which are typically obtained by treatment of fumed silica in the presence of polydimethylsiloxane or dimethyldichlorosilane are known as “silica dimethyl silylate” and are available commercially. For example, under the name “Aerosil R972®”, “Aerosil R974®” (Degussa), or “CAB-O-SIL TS-610®” and “CAB-O-SIL TS-720®” (Cabot).

### Polysaccharides

According to some embodiments, the pharmaceutical compositions of the present invention comprise a polysaccharide. According to some embodiments, the compositions of the present invention may further comprise a monosaccharide compound and/or a disaccharide compound. Non limiting examples of monosaccharides that may be used in the compositions of the invention according to some embodiments include: glucose (dextrose), fructose (levulose), galactose, xylose and ribose. Non limiting examples of disaccharides that may be used in compositions of the invention according to some embodiments include: are sucrose, lactose, and maltose.

The term “polysaccharide” as used herein, refers to polymers formed from about 500 saccharide units linked to each other by hemiacetal or glycosidic bonds and may contain as many as 100,000 saccharide units, or more. The polysaccharide may be either a straight chain, singly branched, or multiply branched wherein each branch may have additional secondary branches, and the monosaccharides may be standard D- or L-cyclic sugars in the pyranose (6-membered ring) or furanose (5-membered ring) forms such as D-fructose and D-galactose, respectively, or they may be cyclic sugar derivatives, for example amino sugars such as D-glucosamine, deoxy sugars such as D-fucose or L-rhamnose, sugar phosphates such as D-ribose-5-phosphate, sugar acids such as D-galacturonic acid, or multi-derivatized sugars such as N-acetyl-D-glucosamine, N-acetylneuraminic acid (sialic acid), or N-sulfato-D-glucosamine. When isolated from nature, polysaccharide preparations comprise molecules that are heterogeneous in molecular weight. Polysaccharides include, among other compounds, galactomanans and galactomannan derivatives; galacto-rhamnogalacturons and galacto-rhamnogalacturon derivatives, and galacto-arabinogalacturon and galacto-arabinogalacturon derivatives.

According to some embodiments, the polysaccharide is a naturally-occurring polysaccharide, a naturally-occurring branched polysaccharide, a synthetic polysaccharide or a synthetic branched polysaccharide. Each possibility represents a separate embodiment of the present invention. Non limiting examples of synthetic polysaccharides are disclosed in U.S. Pat. No. 6,528,497.

According to some embodiments, the polysaccharide is a branched polysaccharide. The term “branched polysaccharides” is well understood to those skilled in the art and may refer to any number and structure of branches in the links between monosaccharide monomers. According to some embodiments, the polysaccharide is a naturally-occurring branched polysaccharide. According to some embodiments, the branched polysaccharide is a starch. According to further embodiments, the branched polysaccharide is a starch derivative. According to some embodiments, the branched polysaccharide is selected from the group consisting of amylopectin, glycogen, and a branched alpha glucan. According to some embodiments, the polysaccharide is a synthetic branched polysaccharide. Each possibility represents a separate embodiment of the present invention.

According to some embodiments, the polysaccharide is an amphipathic polysaccharide. The term “amphipathic polysaccharide” is well understood to those skilled in the art and refers to the existence of both hydrophobic and hydrophilic regions on the polysaccharide. According to some embodiments, the polysaccharide is a naturally-occurring amphipathic polysaccharide. Each possibility represents a separate embodiment of the present invention.

According to some embodiments, the average molecular weight (MW) of the polysaccharide is at least 1 kilodalton (kDa), at least 3 kDa, at least 5 kDa, at least 10 kDa, at least 50 kDa, at least, 100 kilodalton (kDa), at least 150 kDa, at least 200 kDa, at least 300 kDa, at least 400 kDa, at least 500 kDa, at least 600 kDa, at least 800 kDa, at least 1,000 kDa, between 100 to 1,000 kDa, between 150 to 1,000 kDa, between 1 to 800 kDa, between 1 to 500 kDa or between 1 to 300 kDa. Each possibility represents a separate embodiment of the present invention.

According to some embodiments, the polysaccharide is selected from the group consisting of starch, dextrin, cellulose, chitin, a branched alpha glucan, a branched beta glucan and derivatives thereof. According to some embodiments, the polysaccharide comprises a polymer of glucose having the backbone formula (C₆H₁₀O₅)ₙ, including, but not limited to, cellulose, dextrin, starch and glycogen. Each possibility represents a separate embodiment of the present invention.

According to some embodiments, the polysaccharide is a starch. Non-limiting examples of starch include, corn starch, potato starch, rice starch, wheat starch, purum starch, and starch from algae. Each possibility represents a separate embodiment of the present invention. According to some embodiments, the polysaccharide is cellulose. Non-limiting examples of cellulose include α-cellulose and β-cellulose.

According to some embodiments, the polysaccharide is an alpha-glucan. The alpha-glucan may be linear or branched with alpha 1-2, alpha 1-3, alpha 1-4, and/or alpha 1-6 glycosidic linkages. Alternatively, the alpha-glucan may have unbranched linear glucose polymers with 1-4 glycosidic linkages, an example of which is alpha-amylose. Optionally, the alpha-glucan may have branched glucose polymers with alpha 1-4 glycosidic linkages in the backbone and alpha 1-6 linkages at branch points, an example of which is amylopectin. According to some embodiments, the polysaccharide is a beta-glucan.

### Cyclodextrins

According to certain embodiments, the solid particulate ingredients further comprise a cyclodextrin. According to one embodiment, the cyclodextrin is a naturally occurring cyclodextrin selected from the group consisting of α-cyclodextrin, β-cyclodextrin, γ-cyclodextrin or a combination thereof. According to certain preferred embodiments, the pharmaceutical composition of the invention comprises β-cyclodextrin.

### Biopolymers

According to some embodiments, the compositions of the present invention may further comprise a biopolymer. According to further embodiments, the biopolymers comprise branched biopolymers. The term “branched” as used herein refers to polymers that are naturally branched and those engineered to be branched by physical treatment such as thermal and/or ultrasound treatment. In general, branched polymers are defined as polymers wherein a monomer subunit is covalently bound to more than two monomer subunits. Such a monomer is the site of a branch point, wherein multiple polymer chains converge. In another embodiment, the branched biopolymer is a crosslinked polymer. According to some embodiments, the branched biopolymer is not crosslinked Non-limiting examples of branched polymers are glycogen and amylopectin, forms of starch derived from animals and plants, respectively.

According to some embodiments, the biopolymer is a fibrous biopolymer. The term “fibrous polymer” as used herein, refers to a polymer in the form of a network of discrete thread-shaped pieces. Non-limiting examples of fibrous polymers are guar gum (for example, Benefiber™), collagen, keratin, fibrin, and elastin. Biopolymers may be either naturally fibrous or made fibrous by physical and chemical treatment.

According to some embodiments, the biopolymer is a fiber. The term “fiber” as used herein refers to an indigestible component that acts as a bulking agent for feces. The fiber may be an insoluble fiber or a soluble fiber. Each possibility represents a separate embodiment of the present invention. Each type of fiber and type of branched and fibrous biopolymer represents a separate embodiment of the present invention.

According to some embodiments, the biopolymer is pharmacologically and/or biologically inert. According to some embodiments, the biopolymer is non-toxic. According to some embodiments, the biopolymer is non-teratogenic. Each possibility represents a separate embodiment of the present invention.

According to some embodiments, the melting temperature (Tₘ)) of the biopolymer falls within a range particularly suitable for compositions of the present invention, including, a melting temperature under 400° C., below 350° C., below 300° C., below 250° C., below 200° C., below 150° C., between 100-400° C. or any Tₘ falling within a ranges disclosed herein. Each possibility represents a separate embodiment of the present invention.

### Structural Proteins

According to certain embodiments, the solid particulate ingredients of compositions may further comprise a structural protein. The term “structural protein” as used herein commonly refers to a high molecular weight (MW) structural protein, which confers a structure to a cell, cellular membrane, or extracellular membrane in vivo. The structural protein may comprise hydrophilic and hydrophobic residues that interact with the hydrophobic and hydrophilic regions, respectively, of the biologically active protein or peptide. According to certain embodiments, the average MW of the structural protein is at least 100 kilodalton (kDa).

According to certain embodiments, the structural protein is a fibrous protein. According to certain embodiments, the structural protein is a scleroprotein. According to certain embodiments, the structural protein is selected from the group consisting of elastin, collagen, keratin, and fibrinogen. Each possibility represents a separate embodiment of the present invention.

According to some embodiments, the structural protein is having a Tₘ within a range of melting temperatures particularly suitable for compositions of the present invention, such as, a Tₘ under 400° C.

### Oils and Oil Coatings

The particulate matter of oil-based matrix compositions of the present invention is surrounded by, suspended in, immersed in, embedded in or dispersed in oil carrier. Typically, the oil phase, in addition to coating the particulate matter, impregnates the particulate matter, which is composed of the silica particles, branched polysaccharide, insulin, C-Peptide and/or proinsulin. The terms “oil carrier”, “oil,” “oil layer,” “oil phase,” and “oil coating” as used herein are interchangeable and refer to the aforementioned oil surrounding the particulate matter of matrix compositions of the present invention. The oil may further include an additional component or components useful in the compositions and methods of the present invention (e.g. a fat-soluble co-factor or anti-oxidant). The oil is composed primarily of a pharmaceutically acceptable oil carrier, in which the other components are mixed and/or dissolved. The oil carrier may be composed of either one or a plurality of types of oils, as described further herein. According to some embodiments, the coating consists essentially of lipids and/or oils.

According to some embodiments, at least 5% of the composition is oil. According to some embodiments, at least 20% of the composition is oil. According to some alternative embodiments, at least 25%, at least 30%, at least 35%, at least 40%, at least 45%, at least 50%, at least 55% of the composition is oil. Each possibility represents a separate embodiment of the invention. According to other embodiments, at least 60% of the composition is oil. According to further embodiments, at least 65% of the composition is oil.

According to some embodiments, the weight of the particulate matter is not more than 80% of the total weight of the composition. According to various embodiments the weight of the particulate matter of the composition is 25-80% of the total weight of the composition. According to some alternative embodiments the weight of the particulate matter is no more than 70%, not more than 60%, not more than 50%, not more than 40% of the weight of the pharmaceutical composition. According to yet another embodiment, the weight of the particulate matter is at least 35% of the total weight of the composition.

According to some embodiments, the weight ratio of the oil and the particulate matter ranges from 10:1 to 1:20. According to specific embodiments the weight ratio of the oil and the particulate matter is at least 1:4. According to alternative embodiments, the weight ratio of the oil and the particulate matter is at least 1:6. According to alternative embodiments, the weight ratio of the oil and the particulate matter is at least 1:3, 1:2, 1:1.5 or 1:1. According to other embodiments, the weight ratio of the oil and the particulate matter is at least 1.5:1. According to further embodiments, the weight ratio of the oil and the particulate matter is at least 2:1. According to further embodiments, the weight ratio of the oil and the particulate matter is at least 3:1. Optionally, the weight ratio of the oil and the particulate matter ranges from about 1:4 to about 3:1, from about 1:3 to about 3:1, from about 1:2 to about 3:1, from about 1:1.5 to about 3:1, from about 1:1 to about 3:1, from about 1.5:1 to about 3:1, from about 1:1 to about 1:3, from about 1:1.5 to about 1:3, from about 1:2 to about 1:3, from about 1:4 to about 2:1, from about 1:3 to about 2:1, from about 1:2 to about 2:1, from about 1:1.5 to about 2:1, from about 1:1 to about 2:1, from about 1.5:1 to about 2:1, from about 1:4 to about 1:1, from about 1:3 to about 1:1, from about 1:2 to about 1:1 or from about 1:1.5 to about 1:1. Each possibility represents a separate embodiment of the present invention.

According to some embodiments, the oil carrier is a naturally occurring oil. According to some embodiments, the oil is a mixture of natural vegetable oils, such as, sesame oil, olive oil, linseed oil, evening primrose oil, silicone oil, sea buckthorn oil, sunflower oil, corn oil, soybean oil, coconut oil, palm oil, jojoba oil, marrow oil, grapeseed oil, hazelnut oil, apricot oil, macadamia oil and castor oil or combinations thereof.

According to some embodiments, the oil carrier is of animal origin, such as lanolin. According to some embodiments, the oil carrier is a synthetic oil. According to some embodiments, the oil carrier is a fatty alcohol. According to some embodiments, the oil carrier is 2-octyldodecanol. According to some embodiments, the oil carrier is selected from the group consisting of a fatty acid ester, a phenylsilicone, phenyltrimethicone, a diphynyldimethicone and a poly-methylphenylsiloxane. Each possibility represents a separate embodiment of the present invention.

According to some embodiments, the oil consists essentially of naturally-occurring lipids and/or oils. Each possibility represents a separate embodiment of the present invention.

According to some embodiments, the oil consist fatty acids, such as caprylic acids, decanoic acid, etc. Each possibility represents a separate embodiment of the present invention.

According to some embodiments, the oil phase of the matrix carrier composition comprises a plurality of oils.

The term “plurality of oils” as used is herein refers to a combination or a mixture of two or more oils. According to some embodiments, the oil comprises three or more oils or four or more oils. According to some embodiments, the oil comprises more than four oils. According to some embodiments, the oil comprises a mixture of vegetable oils. According to some embodiments, the oil or mixture of oils comprise olive oil or an extract thereof.

Without wishing to be bound by a specific theory or mechanism of action, the oil comprises a component capable of stimulating secretion of bile salts or bile acids when ingested by a subject. The component may be any bile salt/acid stimulating lipid-soluble substance known in the art. Alternatively, the bile-stimulating component may be the oil or the carrier is the bile salt/acid stimulating substance. The bile salt/acid stimulating substance may be a substance separate from the carrier. Each possibility represents a separate embodiment of the present invention.

According to some embodiments, the oil contains a significant quantity of one or more antioxidants. For example, the oil is sea buckthorn (oblepicha), which contains a significant quantity of beta-carotene.

According to some embodiments, the oil may further comprise at least one permeability enhancer selected from a medium chain fatty acid, a polyol or a combination thereof. Without being bound by theory of mechanism of action, medium chain fatty acids and polyols enhance mucous permeability.

According to some embodiments, the oil comprises a component that has a melting temperature (Tₘ) of at least 10° C. According to some embodiments, the high Tₘ component is an oil. According to some embodiments, the carrier is the high Tₘ component. According to some embodiments, the high-Tₘ component is included in addition to the carrier. A non-limiting example of a high-Tₘ oil is jojoba oil. According to some embodiments, the high Tₘ oil is used as the oil carrier. Each possibility represents a separate embodiment of the present invention.

According to some embodiments, the mixture of insulin with proinsulin and/or C-Peptide is included in the additional oil or mixture of oils, or included in the first-added oil or mixture of oils. According to some embodiments, the insulin, C-Peptide and proinsulin are combined with an antioxidant and oil (the first-added or additional oil or mixture of oils) prior to adding to the solid phase. Each possibility represents a separate embodiment of the present invention.

According to some embodiments, the additional oil, oil or mixture of oils have a higher viscosity than the first-added oil or mixture of oils.

Without wishing to be bound by any specific theory or mechanism of action, the use of a higher viscosity oil or oil mixture at this stage enables self-ordering or self-organization of structure due to competitive adsorption and minimization of the free energy.

According to some embodiments, the composition of the present invention further comprises a third oil or mixture of oils, wherein the third oil may further comprise an antioxidant. According to some embodiments, the oil carrier of the third oil is sesame oil. According to some embodiments, the third oil, oil or mixture of oils has a higher viscosity than the additional oil or mixture of oils. Each possibility represents a separate embodiment of the present invention.

According to some embodiments, a highly penetrative oil carrier is included in the oil or mixture of oils. Non-limiting examples of highly penetrative oils are sesame oil, tea tree (Melaleuca) oil, lavender oil, almond oil, and grape seed oil.

Without wishing to be bound by any theory or mechanism of action, the highly penetrative oil carrier promotes efficient transport of the active ingredients into the blood.

According to some embodiments, the pharmaceutical composition of the present invention further comprise a pharmaceutically acceptable wax. The term “wax” as used herein refers to a lipophilic compound, which is solid at room temperature (25° C.), with a reversible solid/liquid change of state, having a melting point of greater than or equal to 30° C., which may be up to 120° C. By bringing the wax to the liquid state (melting), it is possible to render it miscible with any oils present and to form a microscopically homogeneous mixture, but on returning the temperature of the mixture to room temperature, recrystallization of the wax in the oils of the mixture may be obtained. The wax may be a natural wax, for example bees wax, a wax derived from plant material, or a synthetic wax prepared by esterification of a fatty acid and a long chain alcohol. Other suitable waxes include petroleum waxes such as a paraffin wax. The wax may stabilize the pharmaceutical composition. Inclusion of wax may facilitate formation of a tablet containing the pharmaceutical composition.

### Absorption Enhancers

The pharmaceutical compositions of the present invention are highly absorbed into the intestinal mucosa due to their unique composition and structure. Efficient transport through the intestinal mucosa into the blood may be further achieved by inclusion of a highly penetrative oil carrier in the oil phase. Without wishing to be bound by any theory or mechanism of action, it is suggested that the polysaccharide, particularly when branched, absorbs hydraulic and mechanical stresses experienced during digestion. The oil coating constitutes a physical barrier that provides additional protection against digestive enzymes. Secretion of bile acids typically causes dispersion of the oil suspension into smaller particles, which can be absorbed in the small intestine. While the particle size is reduced after traversing the stomach and entering the small intestine, the particles remain in a size range of 30-1000 nm, too large to be a substrate for lipases and peptidases, preserving the protective effect of the composition. Advantageously, lipid-coating particles of this size are absorbed to chylomicrons by lacteal vessels, which are lymphatic vessels originating in the villi of the small intestine. Particles absorbed in this manner can reach the bloodstream without undergoing first-pass metabolism, largely preserving the biological activity of the insulin.

According to additional embodiments, improved absorption and efficient transport through the intestinal mucosa of the pharmacologically active proteins (e.g insulin, proinsulin and/or C-peptide) may be further increased by the addition of at least one absorption enhancer. Non limiting examples of absorption enhancers that may be included the composition of the invention include: bile salts, anionic surfactants, medium-chain fatty acids, phosphate esters and sodium N-[8-(2-hydroxybenzoyl)amino]caprylate.

### Pharmaceutically Acceptable Excipients

The composition of the present invention may further comprise one or more pharmaceutically acceptable excipients some of which are useful for the improvement of the therapeutic effect of the drug and others influencing drug consistence and the final dosage form.

Suitable excipients include: Antifoaming agents (e.g. dimethicone, simethicone); Antimicrobial preservatives (e.g. benzalkonium chloride, benzelthonium chloride, butylparaben, cetylpyridinium chloride, chlorobutanol, chlorocresol, cresol, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Chelating agents (e.g. edetate disodium, ethylenediaminetetraacetic acid and salts, edetic acid); Coating agents (e.g. sodium carboxymethyl-cellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein); Colorants (e.g. caramel, red, yellow, black or blends, ferric oxide); Complexing agents (e.g. ethylenediaminetetraacetic acid and salts (EDTA), edetic acid, gentisic acid ethanolmaide, oxyquinoline sulfate); Desiccants (e.g. calcium chloride, calcium sulfate); Flavors and perfumes (e.g. anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Humectants (glycerin, hexylene glycol, propylene glycol, sorbitol); Polymers (e.g., cellulose acetate, alkyl celluloses, hydroxyalkylcelluloses, acrylic polymers and copolymers); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); This list is not meant to be exclusive, but instead merely representative of the classes of excipients and the particular excipients which may be used in oral dosage compositions of the present invention. Each possibility represents a separate embodiment of the present invention.

According to some embodiments, the pharmaceutical composition of the present invention comprises at least one excipient into which the oil having the suspended particulate matter is mixed. In another embodiment, the excipients include one or more additional polysaccharides. In these embodiments, the weight of the oil may be less than 20% of the weight of the composition. However, the weight ratio between the particulate matter and the oil in these embodiments remains as disclosed hereinabove.

The composition of the present invention may further comprise pharmaceutical-grade stabilizer. Stabilizers are well known in the art, and are described, inter alia, in the Handbook of Pharmaceutical Excipients (eds. Raymond C Rowe, Paul J Sheskey, and Sian C Owen, copyright Pharmaceutical Press, 2005).

### Formulations

According to some embodiments, the pharmaceutical compositions of the present invention comprise insulin, proinsulin and C-Peptide, and an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with insulin, proinsulin and C-Peptide, and wherein the weight ratio of insulin to proinsulin is from about 25:1 to about 1:2, the weight ratio of insulin to C-Peptide is from about 3:1 to about 1:2 and the weight ratio of silica to insulin, proinsulin and C-peptide is from about 100:1 to about 1:1. According to further embodiments, the pharmaceutical compositions of the present invention comprise insulin and proinsulin, and an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with insulin and proinsulin, and wherein .the weight ratio of insulin to proinsulin is from about 25:1 to about 1:2, and the weight ratio of silica to insulin and proinsulin is from about 100:1 to about 1:1. According to yet further embodiments, the pharmaceutical compositions of the present invention comprise insulin and C-Peptide, and an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with insulin and C-Peptide, and wherein .the weight ratio of insulin to C-Peptide is from about 3:1 to about 1:2 and the weight ratio of silica to insulin and C-peptide is from about 100:1 to about 1:1. In these embodiments, the polysaccharide may be selected from the group consisting of starch, a starch derivative, cyclodextrin, amylopectin, glycogen and a combination thereof. The compositions of the present invention may include a branched polysaccharide and/or a linear polysaccharide. The composition of the invention may include a biopolymer, such as, a dietary fiber, also known as “roughage.” The dietary fiber may be an insoluble fiber, a linear insoluble fiber, a soluble fiber or a linear soluble fiber. Thus, the solid phase mixture may include a branched polysaccharide and/or a linear polysaccharide, together with a fiber. Pharmaceutical compositions provided herein comprise at least one of cyclodextrin, preferably beta-cyclodextrin, linear saccharide, such as mannitol and starch, preferably Nutriose® soluble dietary biofiber (Formulations A-D).

The active pharmaceutical ingredients, for example, insulin, C-Peptide and proinsulin, may be included in the additional oil or mixture of oils, rather than in the first-added oil or mixture of oils.

According to some embodiments, the pharmaceutical composition of the present invention may be in a liquid, solid, semi-solid or gel form. According to further embodiments, the pharmaceutical composition is formulated in a dosage form selected from: tablet, gel capsule, a hard gelatin capsule, pills, powders, granules, elixirs, suspensions or syrups. The components may be mixed in a particular order in order to produce oil-coated matrix carrier compositions that protect the active ingredients from digestive processes in the stomach.

According to some embodiments, the final formulation form of the pharmaceutical composition of the invention may include any type of oral formulation form, such as, but not limited to: capsules, microcapsules, tablets, microencapsulated tablets, liquid form, gel form, liquid form coated by gel or a hard phase, and pressured tablet.

According to some embodiments, the tablet may be formulated as a dry-coated tablet. Dry coated tablets are suitable for the delivery of a drug in a pulsatile way, at predetermined times following oral administration. The dry-coated tablet may be prepared by compression process, wherein the dry-coated tablet comprises an inner core and an outer shell. The compression method eliminates the time-consuming and complicated coating or granulation processes and also improves the stability of the drug by protecting it from moisture. According to some embodiments, the inner core of said dry-coated tablet may comprise the pharmaceutical composition according to some embodiments of the invention, while the outer shell may comprise materials useful for improving the stability, solubility and/or taste of the formulation. The outer shell may comprise hydrophobic and/or hydrophilic materials. Non limiting examples of hydrophilic coating include: solutions including treated agar, microcrystal cellulose, lactose and starch. The hydrophilic coating may be further enriched with flavors and taste agents. Furthermore the outer shell may be enriched with enhancers such as vitamins and/or anti-oxidative components; e.g. vitamin C and vitamin K.

Non limiting examples of hydrophobic coating include palm oil based materials, or other oils based materials presented as solid under ambient temperature.

According to some embodiments, a pharmaceutical composition of the present invention is in a form selected from the group consisting of a soft gel capsule, a hard gelatin capsule, tablet, coated tablet, pressured tablet, powder, a suspension and a paste. In some embodiments, the pharmaceutical composition is in a liquid form. In additional embodiments, the pharmaceutical composition may be in the form of small or micro-droplets impregnated into biocompatible soluble porous nutritional material (such as, for example, agar, fruit jelly and cornflakes) or into any biocompatible water based gel. In such case, the composition may further include additional ingredients, such as, but not limited to emulsifiers or surfactants (e.g. lecithin, Tween-20 or Tween-80).

In other embodiments, the pharmaceutical composition may be formulated in a microencapsulated dosage form. “Microencapsulated dosage form”, as defined herein, refers to a dosage form in which small or micro-droplets are surrounded by a solid coating. Without wishing to being bound by any specific theory or mechanism of action, the microencapsulation of the pharmaceutical compositions of the present invention allows increasing the surface area of the particulate matter suspended in the oil carrier by forming small or micro-droplets comprising said suspended particulate matter.

According to some embodiments, the microencapsulated pharmaceutical composition comprises an oil carrier having particulate matter suspended therein and an excipient. According to further embodiments, the excipient is present in the microencapsulated pharmaceutical composition in a weight percent ranging from about 10% to about 80% of the total weight of the composition. According to further embodiments, the excipient is present in a weight percent of from about 20% to about 70% of the total weight of the composition. According to additional embodiments, the excipient is present in a weight percent of from about 30% to about 60% of the total weight of the composition.

According to further embodiments, the microencapsulated pharmaceutical composition is in a tablet or a powder form.

### Methods of Treatment

The present invention provides a method for treating diabetes in a subject in need thereof, comprising orally administering to the subject the pharmaceutical composition of the present invention. According to further embodiments, orally administering to the subject the pharmaceutical composition of the present invention provides normoglycemic control. According to yet further embodiments, orally administering to the subject the pharmaceutical composition of the present invention provides treatment of diseases related to carbohydrates metabolic pathways. According to yet further embodiments, orally administering to the subject the pharmaceutical composition of the present invention allows reducing the dosage of injected insulin. According to still further embodiments, the pharmaceutical composition is administered in combination with lower therapeutic doses of injected insulin, compared to the dose required without orally administered combination of the at least two molecules associated with glucose metabolism in a suitable delivery vehicle. According to yet further embodiments, the pharmaceutical composition is administered in combination with a 30% lower therapeutic dose of injected insulin, compared to the dose required without orally administered combination of the at least two molecules associated with glucose metabolism in a suitable delivery vehicle. According to still further embodiments, the pharmaceutical composition is administered in combination with a 50% lower therapeutic dose of injected insulin, compared to the dose required without orally administered combination of the at least two molecules associated with glucose metabolism in a suitable delivery vehicle. According to yet further embodiments, the pharmaceutical composition is administered in combination with a 70% lower therapeutic dose of injected insulin, compared to the dose required without orally administered combination of the at least two molecules associated with glucose metabolism in a suitable delivery vehicle. According to still further embodiments, the pharmaceutical composition is administered in combination with lower therapeutic doses of injected insulin, compared to the dose required with orally administered insulin in a suitable delivery vehicle. According to additional embodiments, the present invention provides a method for decreasing fluctuations in glucose concentration levels, comprising orally administering to the subject the pharmaceutical composition of the present invention. According to yet another embodiment, the present invention provides a method for treating one or more complications of diabetes in a subject in need thereof, comprising orally administering to said subject the pharmaceutical composition of the invention. According to some embodiments, said diabetes is selected from the group consisting of: Type I diabetes, Type II diabetes and gestational diabetes. Each possibility represents a separate embodiment of the present invention.

According to additional embodiment, the present invention provides a method for treating obesity and/or obesity-related conditions in a subject in need thereof, comprising orally administering to said subject the pharmaceutical composition of the invention. According to yet additional embodiments, the present invention provides a method for treating a metabolic disease or condition other than diabetes or obesity in a subject in need thereof, comprising orally administering to said subject the pharmaceutical composition of the invention. According to some embodiments, the metabolic disease or condition is selected from the group consisting of metabolic syndrome, hyperlipidemia, hypercholesterolemia, hypertriglyceridemia, hyperglycemia, insulin resistance, hepatic steatosis, kidney disease, fatty liver disease and non-alcoholic steatohepatitis. Each possibility represents a separate embodiment of the invention.

As used herein the term “metabolic disease” refers to a group of identified disorders in which errors of metabolism, imbalances in metabolism, or sub-optimal metabolism occur. The metabolic diseases as described herein also include diseases that can be treated through the modulation of metabolism, although the disease itself may or may not be caused by a specific metabolic defect. Such metabolic diseases may involve, for example, glucose and fatty acid oxidation pathways.

The term “obesity” as used herein is defined in the WHO classifications of weight. Underweight is less than 18.5 BMI (thin); healthy is 18.5-24.9 BMI (normal); grade 1 overweight is 25.0-29.9 BMI (overweight); grade 2 overweight is 30.0-39.0 BMI (obesity); grade 3 overweight is greater than or equal to 40.0 BMI. BMI is body mass index (morbid obesity) and is kg/m.sup.2. Waist circumference can also be used to indicate a risk of metabolic complications. Waist circumference can be measured (in cm) at midpoint between the lower border of ribs and the upper border of the pelvis. Other measures of obesity include, but are not limited to, skinfold thickness and bioimpedance, which is based on the principle that lean mass conducts current better than fat mass because it is primarily an electrolyte solution.

The term “obesity-related condition” as used herein refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, diabetes (e.g., type 1 diabetes, type 2 diabetes, and gestational diabetes), Syndrome X, hyperglycemia, hyperinsulinemia, impaired glucose tolerance, impaired fasting glucose, dyslipidemia, hypertriglyceridemia, insulin resistance, hypercholesterolemia, atherosclerosis, coronary artery disease, peripheral vascular disease, and hypertension.

According to some embodiments, the pharmaceutical composition is administered instead of parenterally administered insulin. According to other embodiments, the pharmaceutical composition is administered in combination with reduced doses of parenterally administered insulin. In these embodiments, peroral insulin and parenteral insulin administration can be performed simultaneously or sequentially or on entirely independent separate regimens. For example, the oral pharmaceutical composition may be administered several times a day and parenteral insulin may be administered less frequently or at lower doasages.

The methods of the invention further include combined therapy, where the active ingredients, for example, insulin, C-Peptide and/or proinsulin are provided to a patient in need thereof, in combination with other ingredients, namely, antioxidants, free amino acids, non-insulin glucose lowering drugs, blood pressure lowering drugs, glucagon-like peptides, metabolism influencing agents, related disease treatment agents and/or absorption enhancers. The choice of antioxidants, amino acids non-insulin glucose lowering drugs, blood pressure lowering drugs, glucagon-like peptides and/or absorption enhancers may be designed per patient, based on the disease and other parameters related to the specific patient. Some of the main complications associated with diabetes are:
i. Oxidative stress on kidney, inner organs or placenta;
ii. Amino acids metabolism misbalance;
iii. Increased activity of cytokines;
iv. Retinopathy
v. Lower limb gangrene, and
vi. Insulin resistance.

Thus, the present invention provides custom-made therapy, where the drug combination is personalized as per the patient's needs, biochemistry and physiology. By doing so, the chosen antioxidant, free amino acid or absorption enhancer or any combination thereof helps to compensate metabolic misbalances and thus promote the body as a whole to restore a “healthy” metabolism.

The present invention also relates to methods of using one or more combinations of various formulations in the same patients in order to induce a pulsatile treatment, thereby assisting the body to restore its natural balance.

The physician designing a personal therapy based on the pharmaceutical compositions of the invention and combinations thereof, may use specific tests in order to diagnose specific misbalance. Non-limiting example for such tests include amino acid profile test, C-Peptide test, oxidative stress, lipid peroxidation products, etc.

Additional parameters that may be monitored and evaluated for adjusting the formulations, specific combination of formulations, diet and supplements, include:
i. The molar ratio (valine+leucine+isoleucine):(phenylalanine+tyrosine) may be used for estimating the liver state and for determining and adjusting the dose/level of specific enhancers and supplements.
ii. The level of glucose and lactate with respect to the ratio phenylalanine to tyrosine may be used for estimating the catabolic state.
iii. The ratio of glycine:valine may be used for evaluating protein malnutrition.
iv. The ratio glycine:branched chain amino acids may be used for estimating protein uptake.

The aforementioned metabolic parameters change daily and monthly (before and after a meal, for example). Monitoring hormones, including, sex hormones, insulin, glucose, triglycerides, free fatty acids, glycerol and pyruvates provides useful information for designing the ultimate therapy.

Methods of Preparation of the Pharmaceutical Compositions

According to some embodiments, the present invention provides a method of manufacturing a pharmaceutical composition for oral delivery of insulin, C-Peptide and/or proinsulin, the method comprises the steps of:
(a) blending pharmacologically inert silica particles having a hydrophobic surface, with
   (i) a polysaccharide, and (ii) at least two bioactive proteins selected from the group consisting of insulin, C-Peptide and proinsulin, whereby the silica particles form an non-covalent association with the polysaccharide and with the at least two bioactive proteins; and
(b) mixing the particulate matter (silica particles, polysaccharide, bioactive proteins) into an oil.

In the alternative embodiments, the composition is prepared as follows:
(a) dry blending pharmacologically inert silica particles having a hydrophobic surface, with at least one branched polysaccharide, whereby the silica particles form an intimate non-covalent association with the at least one branched polysaccharide; wherein the silica particles and at least one branched polysaccharide may form a complex.
(b) mixing, dispersing or dissolving at least two bioactive proteins selected from the group consisting of insulin, C-Peptide and proinsulin into an oil; and
(c) mixing the silica particles and at least one branched polysaccharide into the oil, wherein the silica particles, at least one branched polysaccharide, and the proteins are suspended in, embedded in or dispersed in the oil, forming non-covalent association with the at least two bioactive proteins.

In these embodiments, the weight ratio of insulin to proinsulin is from about 25:1 to about 1:2, the weight ratio of insulin to C-Peptide is from about 3:1 to about 1:2 and the weight ratio of silica to insulin, proinsulin and C-peptide is from about 100:1 to about 1:1.

The components are mixed in a particular order, as exemplified herein, in order to produce oil-coated matrix carrier compositions that protect the bioactive proteins from digestive processes in the stomach and small intestine.

The silica particles, polysaccharide, pharmaceutical ingredients, and other optional components (e.g. one or more antioxidants, free amino acids, absorption enhancers) form a matrix that becomes dispersed, embedded or suspended in the oil. The silica particles, polysaccharide, pharmaceutical ingredients, and other optional components may form a complex. Said complex may be dispersed, embedded or suspended in the oil.

It is to be understood that the bioactive proteins, such as, the insulin protein, the C-Peptide and the proinsulin, are non-covalently attached to the hydrophobic surfaces of the silica particles and to the hydrophilic and hydrophobic portions, regions or patches of the surface of the polysaccharide.

According to some embodiments, the insulin, proinsulin and C-peptide are in the form of a dry lyophilized powder which is directly dissolved or dispersed into the oil of step (b). As described herein, a mixture of oils or oil phase will typically comprise an oil carrier. In addition, the mixture of oils or oil phase further comprises an additional oil or oils or an additional component or components.

The step of dry mixing may be performed using a high shear mixer or any other means suitable for generating a homogenous solid phase from silica particles and a branched polysaccharide.

The dry mixing step may further comprise inclusion of an additional biopolymer that is a linear biopolymer, for example, a linear polysaccharide. The additional biopolymer may be a linear high molecular weight structural protein, or a biopolymer selected from the group consisting of chitin, cellulose, a linear alpha glucan, a linear beta glucan, amylose and beta glucan.

The method of manufacturing the pharmaceutical composition of the present invention may further comprise the step of adding an additional oil following the addition of the first-added oil or mixture of oils. The term “additional oil” encompasses an oil or mixture of oils, as described elsewhere herein. As detailed herein, the additional oil component may include an antioxidant.

In some embodiments, the bioactive proteins may be included in the additional oil or mixture of oils, rather than in the first-added oil or mixture of oils.

The method of the present invention may further comprise the step of adding a third oil or mixture of oils after addition of the above-described additional oil or mixture of oils, wherein, the third oil component may further comprise an antioxidant. Each possibility represents a separate embodiment of the present invention.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Formulations for Treating Diabetes

The following formulations presented in tables 1-2 are representative formulations based on the principles of the present invention. The formulations are suitable for treating diabetes as defined hereinabove. The formulations are designed to overcome the problems associated with diabetes, such as, oxidative stress on the kidneys, inner organs or placenta; amino acids metabolism misbalance; increased activity of cytokines and insulin resistance.

TABLE 1
Formulation A

| Materials Name | Quantity, g |
|---|---|
| Insulin | 0.1 |
| C-peptide | 0.1 |
| Proinsulin | 0.1 |
| Silica R972 | 6 |
| *Nutriose ® | 20 |
| Beta-Cyclodextrin | 1.5 |
| Mannitol (Pearlitol) | 1 |
| L-Arginine | 1 |
| Olive oil | 15 |
| Oblepicha oil | 35 |
| Coconut oil | 15 |

*Commercially available Nutriose ® comprises a dextrin resulting from a processed corn starch or wheat starch.

TABLE 2
Formulation B

| Materials Name | Quantity, g |
|---|---|
| Insulin Biocon | 0.1 |
| C-peptide | 0.1 |
| Proinsulin | 0.1 |
| Silica R972 | 6 |
| Cordyceps | 20 |
| Beta-Cyclodextrin | 1.5 |
| Mannitol (Pearlitol) | 1 |
| L-Arginine | 1 |
| Olive oil | 15 |
| Oblepicha oil | 30 |
| Coconut oil | 15 |
| Caprylic acid | 5 |

The proposed formulations are directed to treating diabetes as a group of systematic diseases involving different metabolic misbalances. Therefore, in addition to pancreatic enzymes, namely, a mixture of insulin, proinsulin and C-Peptide, endogeneous as well as exogeneous anti-oxidants, co-factors and free amino acids may be included in the treatment in order to balance metabolic misbalances and thus to promote the body as whole to restore a normal, healthy, metabolism.

The compositions can be formulated in liquid dosage forms and microencapsulated dosage forms. Microencapsulated dosage forms included 55-70% (w/w) excipient.

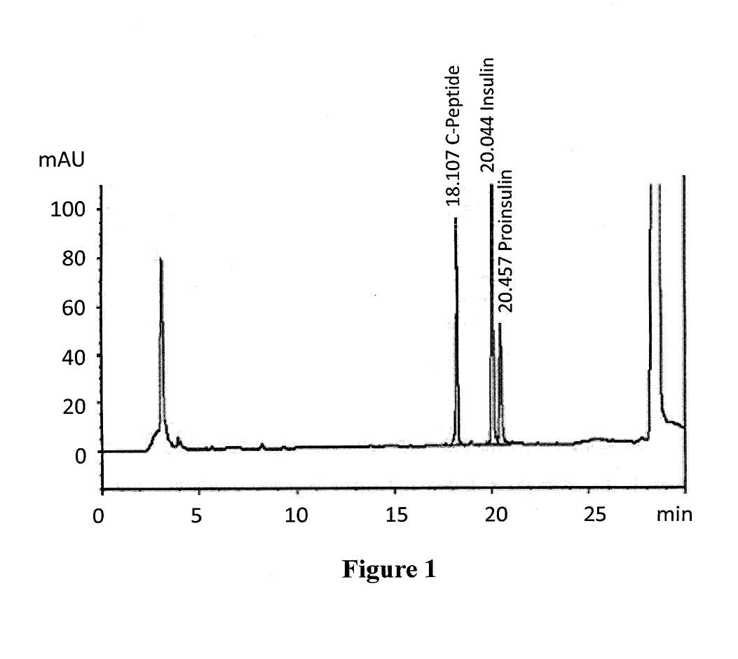

An HPLC chromatogram of Formula A exhibits peaks corresponding to insulin, proinsulin and C-Peptide (FIG. 1).

### Example 2

### Formulations for Treating Diabetes During Pregnancy and for Treating Diabetes Associated with Obesity and Other Complications

The following formulation is a representative formulation based on the principles of the present invention. The formulation is suitable for treating diabetes, and is particularly suitable for treating diabetes in pregnant women.

TABLE 3
Formulation C

| Material name | Quantity, g |
|---|---|
| Proinsulin | 0.05 |
| Insulin | 0.1 |
| C-peptide | 0.05 |
| Silica R972 | 5 |
| Nutriose ® | 20 |
| Beta-cyclodextrin | 1.5 |
| Mannitol | 1 |
| Glutathione | 0.5 |
| Leucine | 0.5 |
| Histidine | 0.5 |
| Olive oil | 20 |
| Oblepicha oil | 25 |
| Coconut oil | 10 |
| Decanoic acid | 10 |

The following formulation is a representative formulation based on the principles of the present invention. The formulation is suitable for treating diabetes, and is particularly suitable for treating diabetes associated with obesity and other complications.

TABLE 4
Formulation D

| Materials Name | Quantity, g |
|---|---|
| Proinsulin | 0.1 |
| Insulin | 0.1 |
| C-peptide | 0.06 |
| Silica R972 | 6 |
| Nutriose ® | 20 |
| SOD | 0.1 |
| Mannitol | 1 |
| Biotin | 1 |
| Leucine | 1 |
| Histidine | 0.5 |
| Vitamin B6 (pyridoxin) | 0.5 |
| Vitamin D | 0.01 |
| Olive oil | 10 |
| Oblepicha oil | 30 |
| Coconut oil | 15 |
| Caprylic acid | 5 |
| Decanoic acid | 5 |

The compositions can be formulated in liquid dosage forms and microencapsulated dosage forms. Microencapsulated dosage forms included 55-70% (w/w) excipient.

### Example 3

### Microencapsulated Formulations

Microencapsulated formulation may be obtained by mixing of the oil-based composition with polysaccharides, for example, HPMC/Hypromellose/hydroxy propyl methyl cellulose to obtain a coating of about 30 μm thickness, wherein the polysaccharide is added in an amount from 15% to 360% (w/w) according to following table:

TABLE 5
Microencapsulated formulation

| | | | Weight of |
|---|---|---|---|
| | | | coating, |
| Diameter, | | | g per g of |
| microns | No/g | Surface, cm² | formulation |
| 50 | 1.5 · 10⁷ | 1200 | 3.6 |
| 100 | 1909859 | 600 | 1.8 |
| 150 | 565884 | 400 | 1.2 |
| 200 | 238732 | 300 | 0.9 |
| 250 | 122231 | 240 | 0.72 |
| 300 | 70735.5 | 200 | 0.6 |
| 350 | 44544.8 | 171.43 | 0.51 |
| 400 | 29841.6 | 150 | 0.45 |
| 450 | 20958.7 | 133.33 | 0.4 |
| 500 | 15278.9 | 120 | 0.36 |
| 550 | 11479.2 | 109.1 | 0.33 |
| 600 | 8841.94 | 100 | 0.3 |
| 650 | 6954.43 | 92.31 | 0.28 |
| 700 | 5568.1 | 85.71 | 0.26 |
| 750 | 4527.07 | 80 | 0.24 |
| 800 | 3730.19 | 75 | 0.23 |
| 850 | 3109.89 | 70.59 | 0.21 |
| 900 | 2619.83 | 66.67 | 0.2 |
| 950 | 2227.57 | 63.16 | 0.19 |
| 1000 | 1909.86 | 60 | 0.18 |
| 1050 | 1649.81 | 57.14 | 0.17 |
| 1100 | 1434.91 | 54.55 | 0.16 |
| 1150 | 1255.76 | 52.17 | 0.16 |
| 1200 | 1105.24 | 50 | 0.15 |

### Example 4

### Dissolution Test for the Pharmaceutical Composition According to Some Embodiments of the Invention

Drug dissolution testing is routinely used to provide critical in vitro drug release information that can be related to in vivo pharmacokinetic data by means of in vitro-in vivo correlations. The dissolution testing included HPLC analysis of the API dissolved in sampled dissolution media at various time points as well as analysis on API present in un-dissolved remains of API that were collected at the end of the dissolution experiment.

The dissolution test was initially performed under conditions which resemble the conditions within the stomach (pH=1.2). Samples were agitated for 2 hours at 75 rpm. After 2 hours of exposition to stomach acidic medium dissolution media was replaced with phosphate buffer at pH 6.8, which serves to imitate the conditions in the small intestine and the samples were agitated for additional 22-24 hours.

### Dissolution Testing Conditions:

Apparatus: Standard Dissolution vessel with stainless steel pedal.
Dissolution test media: Physiologic conditions reflecting the gastrointestinal tract environment:
Media I: 0.1N HCl (pH=1.2)
Media II (gastric fluid simulation): 2 g/L NaCl; 3.2 g/L Pepsin and 0.06 M HCl.
Media III: 0.2 M sodium phosphate buffer (pH 6.8).

### Media IV: FeSSIF (Fasted State Simulated Intestinal Fluid)

pH range: 1.2 to 6.8.
Medium volume: 500 mL.

### Temperature: 37±0.5° C.

Agitation: 50 rpm during 5 min and followed by agitation at 75 rpm.
Dissolution sampling time points:
Media I: dissolution measured at 0, 30, 60, 90 and 120 minutes.
Media II: dissolution measured at 0, 30, 60, 90 and 120 minutes.
Media III: dissolution measured at 0, 1, 2, 4, about 16 and 24 hours.
Media IV: dissolution measured at 0, 1, 2, 4, about 16 and 24 hours.
Criterion: Visual disintegration of the capsule, HPLC analysis.

The dissolution testing results are summarized in Table 6.

TABLE 6
Dissolution testing results

| First | Second | | | C- | API detection |
|---|---|---|---|---|---|
| Medium | Medium | Insulin, | Proinsulin, | Peptide, | in dissolution |
| (2 h) | (22-24 h) | % | % | % | media, % |
| pH = 1.2 | — | 89 | 71 | 92 | Not detected |
| | pH = 6.8 | 92 | 78 | 106 | Not detected |
| | buffer | | | | |
| | pH = 6.8 | 94 | 83 | 106 | Not detected |
| | buffer | | | | |
| pH = 1.2 | pH = 6.8 | 90 | 88 | NA | Low |
| | buffer | | | | |
| pH = 1.2 + | pH = 6.8 | 85 | 82 | NA | Low |
| pepsin | buffer | | | | |
| pH = 1.2 | FeSSIF | 73 | 72 | NA | NA |
| pH = 1.2 + | FeSSIF | 88 | 83 | NA | NA |
| pepsin | | | | | |

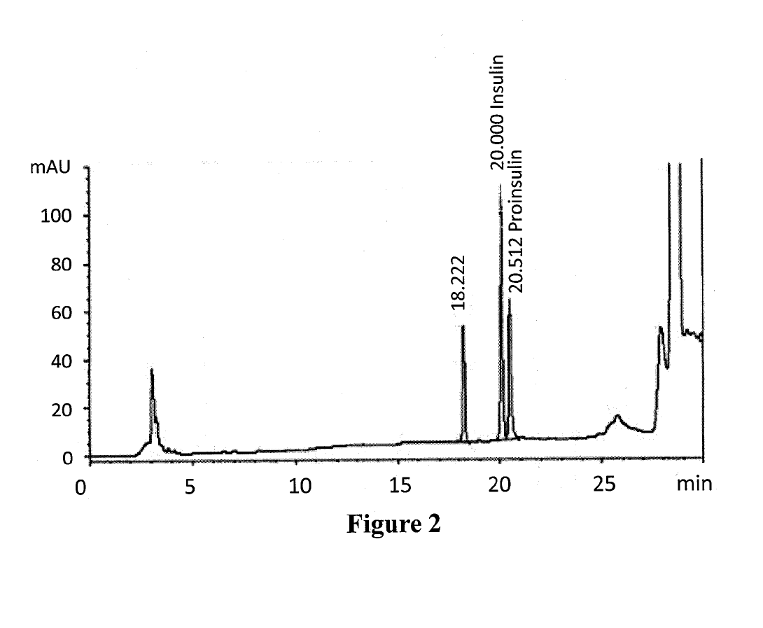

The dissolution testing results summarized in Table 6 indicate that more than 70% of active agents (insulin, proinsulin and C-peptide) remain intact after 2 hours of dissolution under acidic conditions in the presence of pepsin, imitating the harsh conditions inside the stomach followed by 22-24 hours of dissolution at pH=6.8 conditions, imitating the conditions at the small intestine. FIG. 2 shows a HPLC chromatogram of formulation A after a 24 hours of dissolution under the conditions described above, indicating that the majority of the API remained intact.

This observation strongly indicates that the bioactive proteins are protected within the structured complex or matrix formed by at least the silica nanoparticles having hydrophobic surface, polysaccharide/s and oil or mixture of oils.

### Example 5

### Disintegration Test for the Pharmaceutical Composition According to Some Embodiments of the Invention

Disintegration test determines whether tablets and capsules disintegrate within a prescribed time when placed in an immersion fluid under prescribed experimental conditions. Disintegration is defined as the state in which no residue of the tablet or capsule, except fragments of un-dissolved coating or capsule shell, remains on the screen of the test apparatus or, if any other residue remains, it consists of a soft mass having no palpably firm, unmoistened core.

In order to determine the disintegration properties of the compositions of the invention, two capsules of Formulation A were placed in a disintegration test apparatus (Erweka ZT-31) in water at a temperature of 37±2° C. The time required for the capsule to lose the original form until no residue was measured, and the value of the each capsule was given as the disintegration time (Table 7).

TABLE 7
disintegration time of capsules comprising the pharmaceutical
composition of the invention

| Formulation | Media | Leakage time, min | Disintegration time, min |
|---|---|---|---|
| Placebo | water | NA | 9; 9 |
| Placebo | water | 0:31; 0:32 | 7:10; 7:10 |
| Placebo | water | 0:20; 0:22 | 7:15; 8:00 |
| Placebo | buffer | 0:40; 0:42 | 11:50; 12:00 |
| Placebo | buffer | 0:46; 0:41 | 9:35; 9:27 |
| Placebo | water | 0:45; 0:41 | 8:40; 8:19 |
| Placebo | GSF | NA | 5:55; 6:20 |
| Formulation A | water | 0:30; 0:43 | 7:45; 7:50 |
| Formulation A | water | 1:03; 1:02 | 6:30; 7:00 |
| Formulation A | buffer | 0:40; 0:42 | 7:00; 7:10 |
| Formulation A | buffer | 0:45; 1:02 | 9:15; 10:00 |

### Example 6

### Phase I Clinical Study of the Pharmaceutical Composition According to Some Embodiments of the Invention for the Treatment of Type 1 Diabetes Patients

A randomized, multiple-dose, double-blind, placebo-controlled, cross-over study in Type 1 diabetes patients was performed. The study included 2 periods of 3 consecutive days of multiple-dose administration of an oral formulation comprising insulin, C-peptide and pro-insulin (“ICP” or “Oshadi ICP” herein after) according to some embodiments of the invention or placebo for the determination of the safety and pharmacodynamics effect of the oral formulation.

Patients were administered with ICP or placebo (according to a randomization schedule) for three consecutive days. Reduced dose of long and rapid insulin was administered subcutaneously in parallel. After a 12 days washout period, the same procedure was repeated using the alternative administration (ICP or placebo). Patient's blood glucose level was monitored by Continuous Glucose Monitoring System, and at least 10 times a day by finger tips pricking. Ketones were measured 4 times a day by capillary blood sample before meals and before bed time.

Patients were followed 5 and 10 days following the first three days administration sessions and 5 days following the second (and last) three days administration session for drug safety evaluation. Table 8 summarizes the study procedure.

TABLE 8
study procedure:
STUDY PROCEDURE

| | | | | | | |
|---|---|---|---|---|---|---|
| Transfer from | X | | | | | |
| pump to | | | | | | |
| injection | | | | | | |
| Connecting to | | X | | X** | | |
| glucose | | | | | | |
| monitoring | | | | | | |
| system | | | | | | |
| Disconnecting | | | | X* | | X |
| from glucose | | | | | | |
| monitoring | | | | | | |
| system | | | | | | |
| Oshadi | | | X | | X | |
| insulin/ | | | | | | |
| placebo | | | | | | |
| adminis- | | | | | | |
| tration | | | | | | |
| Follow-up | | | | X | | X |
| visits | | | | | | |
| End of study | | | | | | X |
| Time line | 1 week | 1 day | Days | Days | Day | Day |
| | prior to | prior to | 1-4 | 9, 14 | 15-18 | 23 |
| | drug | drug | | | | |
| | adminis- | adminis- | | | | |
| | tration | tration | | | | |
| Duration | 1 | 1 | 3 | 1 | 3 | 1 |
| | hour | hour | nights | hour | nights | hour |
| | | | staying | | staying | |

*Day 9
**Day14

### Research Design and Methods

### Subjects

The study included volunteers above 18 years of age with Type 1 Diabetes Mellitus T1DM (according to ADA criteria) for more than 1 year. ADA criteria are as follows:

1) A1C≧6.5 percent; or

2) Fasting plasma blood glucose ≧126 mg/dL (7.0 mmol/L); or

3) Two-hour plasma glucose ≧200 mg/dL (11.1 mmol/L) during an oral glucose tolerance test (The test should be performed as described by the World Health Organization, using a glucose load containing the equivalent of 75 g anhydrous glucose dissolved in water); or

4) In a patient with classic symptoms of hyperglycemia or hyperglycemic crisis, a random plasma glucose ≧200 mg/dL (11.1 mmol/L).

Exclusion criteria included any other chronic or concurrent disease, except for controlled hypothyroidism.

### Study Procedure:

10 evaluable T1DM patients participated in the study. Subjects were provided with identical low carbs but normal calorie diet (70 gram carbs/day, 1700 or 2300 kcal/d) during both 3 days sessions. The specific diet per patient was chosen by the patients from a designated food list, prior to study initiation. The subjects ingested identical meals during both 3 days sessions, and maintained same level of physical activity during both sessions.

During ICP administration session each patient received a capsule, comprising a fixed dose (not related to the patient's weight or routine insulin administration) of 50 IU insulin, 2 mg proinsulin and 2 mg C-peptide inside Oshadi ICP formulation, 3 times a day (altogether 150 IU/day insulin; 6 mg/day proinsulin and 6 mg/day C-peptide). During placebo administration session each patient received a capsule, comprising Oshadi carrier composition, excluding the active proteins, such that the placebo capsule looked exactly the same as the ICP capsule.

Patients were administrated with half the usual dose of injected long acting insulin and approximately half the usual dose of injected rapid acting insulin during both three days sessions. Long acting insulin was administrated before bedtime at fixed half dose. Rapid acting insulin was injected 5 times a day prior to meals. The half dose of rapid insulin was calculated according to individual insulin carbohydrate ratio and correction factor, based on meal content and patient's pre-meal blood glucose (measured by meter). Meals were identical in both sessions; however, the pre-meal blood glucose concentration levels (measured by meter) were not identical. Therefore, the half dose of the rapid acting insulin doses varied among the matching days. In addition, patients were provided with rescue insulin when blood glucose exceeded a pre-defined level.

Glucose level was monitored by a Continue Glucose Monitoring System (CGMS) in addition to finger prick test capillary glucose concentrations. Mean glucose level and Area under the Curve (AUC) as well as injected insulin doses on the matching days of both sessions were analyzed and compared. Glucose level values during placebo administration were multiplied by an insulin adjustment factor calculated according to the discrepancy between the doses of the insulin injected on the matching days.

### Results

### Safety

No adverse events and no clinically relevant changes in vital signs, electrocardiograms, or in standard safety laboratory parameters were observed throughout the study.

### Episodes of Low Glucose

Although patients were administered with half routine dose of injected insulin, 4 cases of low glucose concentration (<80 mg/dL, measured by meter, 2 in the hypoglycemic range ≦70 mg/dL) were detected during the study, all cases occurred during the Oshadi ICP administration sessions. All hypoglycemic episodes were short and easily controlled with oral carbohydrates administration.

### Efficacy

### Injected Insulin Dose:

Mean rapid acting insulin dose, at the third day of the session, was significantly lower during Oshadi ICP administration session compared to placebo (10.56±3.28 IU insulin ver.13.22±5.4 IU insulin; respectively). Results were found to be statistically significant (p<0.01). Thus, patients were administered with 25% lower doses of rapid acting insulin during Oshadi ICP session, compared to placebo.

### Glucose Concentration:

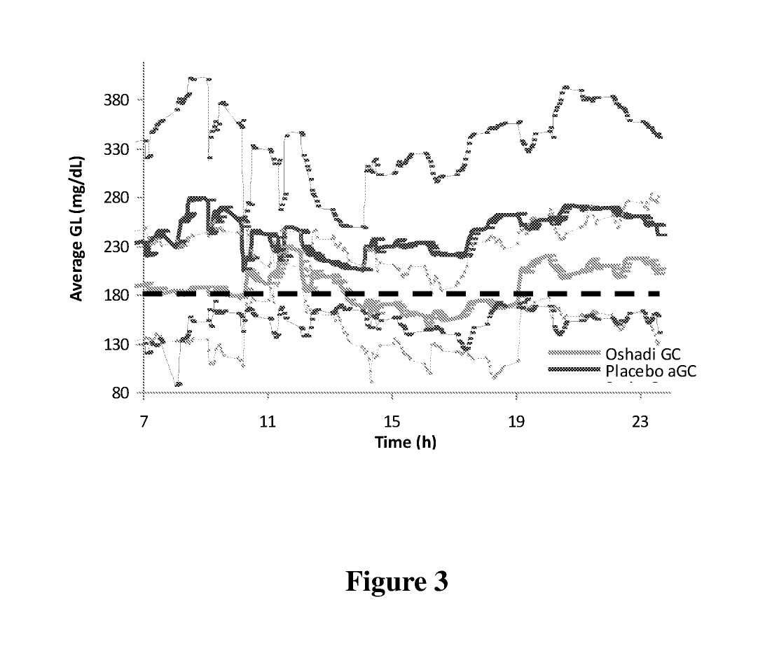

Mean daytime glucose concentration values, at the third administration day, were significantly lower during Oshadi ICP session compared to placebo (187.56±19.48 mg/dL vs 242.14±19.20 respectively). Results were found to be statistically significant (p<0.001). FIG. 3 represents mean GC (glucose concentration) over daytime (7:00-24:00) during the third day of Oshadi ICP administration (Oshadi GC) and during the third day of placebo administration GC (aGC), wherein aGC is adjusted according to the rapid insulin injected dose. The black dotted line indicates GC of 180 mg/dL; STDV is represented by the tiny lines.

### Glucose Area Under the Curve (AUC):

Daytime AUC during Oshadi ICP session, at the third administration day, was significantly lower compared to the third placebo day (3107.57±20.5 vs 4031.58±26.76 respectively, p<0.001).

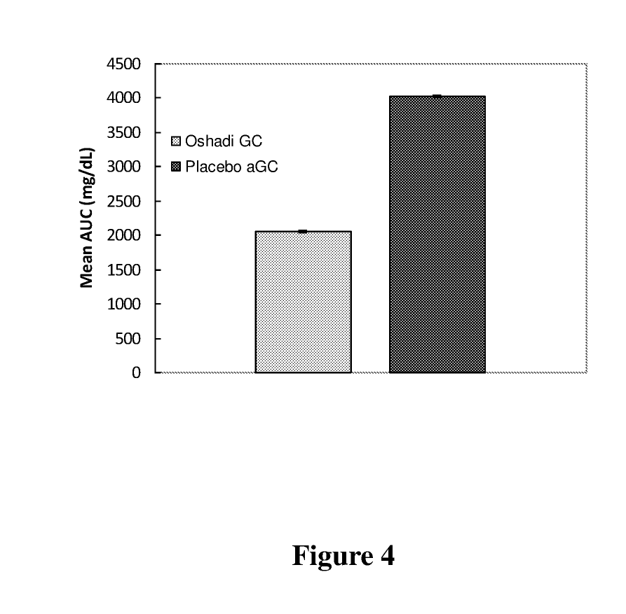

AUC for daytime High Blood Glucose Index (HBGI)—GC>180 mg/dL during Oshadi ICP session, at the third administration day, was significantly lower compared to the third placebo day (2056.78±20.81 vs 4031.58±26.76 respectively, p<0.001). This result indicates that the mean placebo aAUC≧180 level was almost double compared to mean AUC≧180 during the Oshadi ICP session. This result is beyond the insulin adjustment factor applied to placebo GC level, indicating less fluctuation in GC level while administrating Oshadi ICP. FIG. 4 represents mean daytime AUC GC>180 mg/dL.

### Conclusions

This study demonstrated the safety and the glucose lowering effect of Oshadi ICP formulation. The combination of insulin, proinsulin and C-peptide in Oshadi carrier, delivered orally through the portal system, allowed a reduction in the needed injected insulin dose; led to better control of glucose concentration and reduced the fluctuation in glucose concentration levels.

### Example 7

### Phase II Clinical Study of the Pharmaceutical Composition According to Some Embodiments of the Invention for the Treatment of Type 1 Diabetes Patients

This study is a multiple-dose, open-label non-randomized study in patients with Type 1 diabetes, with periodic dose adjustments. The study includes 4 weeks of multiple-dose administration of the oral pharmaceutical composition including insulin, proinsulin and C-Peptide in an oil-matrix carrier, according to some embodiments of the invention (Oshadi ICP), at home and in study center for the determination of the efficacy, safety and pharmacodynamic effects of Oshadi ICP.

Following 1 week of glucose concentration monitoring under routine insulin regiment at home, patients are administered with Oshadi ICP for 4 consecutive weeks in addition to reduced dose (compared to routine use) of subcutaneous (SC) insulin therapy. Oshadi ICP dose is adjusted according to protocol criteria after 2 weeks of administration. Patients are monitored with Continuous Glucose Monitoring System (CGMS) in addition to finger pricks for capillary glucose assessment. Patients are scheduled for follow-up visits once a week and phone follow-ups will be performed daily. At the end of 4 weeks Oshadi ICP administration, patients will return to their routine insulin regiment and will be monitored for blood glucose level for additional 3 weeks

Objectives of the Study
To evaluate the safety and tolerability of multiple doses of Oshadi ICP in type 1 diabetes patients;
To assess the Oshadi ICP effect on glucose levels and glucose variability.
To assess the glycemic control of Oshadi ICP by analyzing average glucose concentration values and variability, fructosamine and HBA1C levels
To assess the effect of Oshadi ICP on High Blood Glucose Index (HBGI).
To evaluate the effect of Oshadi ICP on the total daily injectable insulin requirements.

Design

Patients' glucose concentration is monitored 1 week at home, under routine insulin regiment. Patients' blood glucose level is monitored by Continuous Glucose Monitoring System, and at least 4 times a day by capillary blood sample. Patients are scheduled for one day hospitalization for monitoring glucose and insulin levels under controlled conditions (diet and activity) in the first day of that week.

Patients are scheduled for 2 days hospitalization at the beginning of the 2nd week. During these days, patients are administered with the Oshadi ICP oral insulin in parallel to a reduced dose of injected insulin. Prior to discharge, patients are provided with Oshadi ICP capsules to be taken at home, according to the prescribed daily dosage. In addition, patients are administered with reduced subcutaneous insulin, according to physician instructions.

Patients are scheduled for follow-up visit once a week. In addition, patients are followed daily over the phone. Subcutaneous insulin dose is adjusted according to the desired glucose levels, while the ICP dosing remains constant.

Patients are scheduled for additional 2 days hospitalization after two weeks of Oshadi ICP administration for Oshadi ICP dose adjustment. Patients continue with the ICP administration, in parallel to reduced injected insulin dose for additional 2 weeks. At the end of the Oshadi ICP administration session (altogether 4 weeks) patients are scheduled for additional 2 days hospitalization for glucose and insulin monitoring under controlled conditions. Patients return to their routine subcutaneous insulin therapy regiment prior to discharge. Patients' glucose concentration is monitored for additional 3 weeks under their routine insulin regiment at home. Patients are scheduled for additional 1 day hospitalization at the last follow-up visit, for glucose and insulin monitoring under controlled conditions (diet and physical activity). Study procedure is represented schematically in Table 9.

Oshadi ICP and S.C. insulin doses are determined by the investigator according to patient individual factors. Oshadi ICP capsules contain 150 IU insulin; 6 mg proinsulin and 6 mg C-peptide in Oshadi Carrier (Oshadi Oral ICP) or 75 IU insulin; 3 mg proinsulin and 3 mg C-peptide. Dose should be administered 1.5 hour before meal with 240 cc water.

TABLE 9
study procedure

| | STUDY PROCEDURE | | | | | | | | | | | | |
|---|---|---|---|---|---|---|---|---|---|---|---|---|---|
| Connecting | X | | | | | | | | | | | | |
| to CGMS | | | | | | | | | | | | | |
| Hospitalization | | X | | X | | | X | | | X | | | X |
| Glucose | | X | X | | X | X | | X | X | | X | X | |
| concentration | | | | | | | | | | | | | |
| monitoring, | | | | | | | | | | | | | |
| routine insulin | | | | | | | | | | | | | |
| regiment at home | | | | | | | | | | | | | |
| Oshadi ICP | | | | X | X | X | X | X | X | X | | | |
| administration | | | | | | | | | | | | | |
| Oshadi ICP dose | | | | | | | X | | | | | | |
| adjustment | | | | | | | | | | | | | |
| Follow-up visits | | | | | | X | | | X | | | X | |
| during Oshadi | | | | | | | | | | | | | |
| ICP | | | | | | | | | | | | | |
| administration | | | | | | | | | | | | | |
| Daily phone | | | | | X | X | | X | X | | X | | |
| follow-ups | | | | | | | | | | | | | |
| Disconnecting | | | | | | | | | | | | | X |
| glucose | | | | | | | | | | | | | |
| monitoring | | | | | | | | | | | | | |
| system | | | | | | | | | | | | | |
| End of study | | | | | | | | | | | | | X |
| Time line | Day | Day | Day | Day | Day | Day | Day | Day | Day | Day | Days | Day | Day |
| | 1 | 1 | 2-7 | 8-9 | 10-21 | 15 | 22-23 | 24-35 | 29 | 36-37 | 38-59 | 48 | 60 |
| Duration | 3 | 1 | 6 | 2 | 11 | 3 hour | 2 | 11 | 3 hours | 2 | 12 | 3 | 1 |
| | hours | day | days | days | days | (every | days | days | (every | days | days | hours | day |
| | | | | | | FU | | | FU | | | | |
| | | | | | | visit) | | | visit) | | | | |

Eligibility Criteria

Inclusion Criteria
1. Type 1 diabetes mellitus (according to ADA criteria) for more than 3 year.
2. Male/female 21 years old and older.
3. BMI≧18.5 and ≦25
4. Female of childbearing age must commit to avoid pregnancy and use contraception during the study.
5. Patients must understand and be willing to give written informed consent prior to any study procedures or evaluations and be willing to adhere to all study schedules and requirements.

Exclusion criteria included any other chronic or concurrent disease, except for controlled hypothyroidism.

Concomitant Medications

Concomitant treatment with corticosteroids, therapeutic anticoagulation (Warfarin, Heparin or Low Molecular Weight Heparin), any derivatives of valproic acid, lipid/cholesterol lowering drugs, or any glucose lowering drugs therapy (other than the planned treatment in the protocol) is prohibited during the study.

The use of all prescription, over-the-counter, or herbal medications during the study is recorded in the CRF. Over the counter medications (e.g., acetaminophen for minor pain) are permitted but must be communicated to the study center during each visit.

Subjects may continue on prescribed medications (including routine vitamins, aspirin, and anti-hypertensive drugs) at study entry provided that 1) these medications are not disallowed and are not listed in the exclusion criteria, 2) the medication has been used for at least 2 months so that the adverse event profile is not confused with that of Oshadi oral Insulin, 3) the dosage has not changed within 1 month prior to start of the study. At the discretion of the Investigator, the patient may be treated with medications otherwise prohibited by the protocol as long as there is no impact of the medication on glycemic control. Any such event should be reported to the Medical Monitor in a timely fashion.

Sample Size

12 evaluable Type 1 patients participate in the study. Additional patients may be enrolled to replace patients who discontinue study prematurely for reasons unrelated to safety or tolerance of Oshadi ICP or exacerbation of the underlying disease.

Dose Adjustment of Insulin

Different patients use different insulin regimens to reduce blood glucose level. Oshadi ICP and SC insulin therapy doses are determined by the investigator.

Oshadi ICP initial dose is 150IU, 6 mg proinsulin and 6 mg C-peptide in an oil-based matrix administrated 3 times per day. Oshadi ICP dose may be adjusted after 2 weeks of administration.

At the beginning of Oshadi ICP administration, injected insulin doses are adjusted. Basal insulin doses are reduced to 70% of patient's routine basal insulin. Bolus insulin dose is also reduced to 70% of recommended dose according to meal carbohydrate counting, pre-prandial glucose level and individual correction factor. Unreduced doses (100%) of injected insulin are administered to the patients to target of 100 mg/dl during the day and to target of 150 mg/dl during the night according to individual correction factor if the following hyperglycemic events occurred:

Blood glucose level, measured by meter, >300 mg/dl
Ketones >=1.0 mmol/L+blood glucose >250 mg/dl;

During the first 2 weeks of Oshadi ICP administration (days 8-22) injected insulin doses are adjusted according to glucose levels. Target treatment is an average glucose concentration of 130 mg/dl per day.

At the second two days hospitalization (days 2-23), Oshadi ICP dose augmentation (by 50%-100%) is considered, based on daily S.C. insulin dose during days 15-22. Oshadi ICP dose augmentation depends upon investigator discretion.

Bolus insulin dose is reduced by additional 20% (on top of the 30% dose reduction upon Oshadi ICP administration) as compared to the recommended dose, following each hypoglycemic event.

Basal insulin dose is reduced by additional 20% (on top of the 30% dose reduction upon Oshadi ICP administration) at specific meals if glucose concentration levels are lower than 100.

Every episode of positive ketones or hypoglycemia is reported to the investigator.

The above instructions apply throughout the 4 weeks of Oshadi ICP administration.

Assessments

Safety

The following assessments are used to evaluate the safety of Oshadi ICP administration:
Adverse events, serious adverse events; and
Laboratory abnormal results (liver and kidney functions, electrolytes etc.).

Time frame for safety assessment of Oshadi ICP administration: end of study (Day 60).

Efficacy

The following assessments are used to evaluate the glucose lowering effect of Oshadi ICP administration:
Evaluation of the total daily injectable insulin dose at the hospitalization days: days 1 (standard insulin regiment); days 36-37 (last 2 days of Oshadi ICP administration); and day 60 (after Oshadi ICP washout). Insulin doses (basal and bolus) are compared.
Evaluation of the glucose concentration levels at the hospitalization days: days 1 (regular insulin regiment); days 36-37 (last 2 days of Oshadi ICP administration); and day 60 (after Oshadi ICP washout). Glucose concentration levels are compared.
Comparison of glucose AUC during daytime, postprandial, and HBGL AUC>180 mg/dL at days 1, 36-37 and days 59-60.
Fructosamine and HbA1c levels at days 1, 37 and 60 are compared.

Time frame for assessment of glucose lowering activity and of Oshadi ICP administration: at the end of Oshadi ICP administration session (Day 37).

Pharmacodynamics—Drug Effect Data

The following additional variables are used for the pharmacodynamics evaluation of Oshadi ICP effect:
Area under the curve (AUC) of glucose concentration levels while administrating Oshadi ICP; and
AUC>180 mg/dL while administrating Oshadi ICP.

Pharmacodynamic parameters are calculated from the glucose concentration levels obtained by the Continuous Glucose Monitoring System and correlated with the data obtained by capillary blood samples.

Statistical Methods

Safety

Safety analyses are performed and all adverse events and abnormal laboratory values are assessed according to a standard grading system that is provided. All safety analyses are performed on the intent to treat population (all patients having received at least one dose of Oshadi ICP and having at least one post baseline safety measurement). All data is reported in individual patient listings.

Pharmacodynamics

Noncompartmental pharmacodynamic methods are used to determine the pharmacodynamic parameters of Oshadi ICP, which include AUC.

### Example 8

### Phase I Clinical Study of the Pharmaceutical Composition Comprising Insulin as the Sole Bioactive Protein for the Treatment of Type 1 Diabetes Patients

A randomized, multiple-dose, double-blind, placebo-controlled, cross-over study in Type 1 diabetes patients is performed. This comparative study includes 2 periods of 3 consecutive days of multiple-dose administration of an oral formulation comprising insulin or placebo for the determination of the pharmacodynamics effect of the oral formulation and comparing it to the pharmacodynamics of the ICP formulation.

An exemplary orally administrable formulation comprising insulin is presented in table 10.

Patients are administered with the oral insulin composition or placebo (according to a randomization schedule) for three consecutive days. Reduced dose of long and rapid insulin is administered subcutaneously in parallel. After a 12 days washout period, the same procedure is repeated using the alternative administration (oral formulation or placebo). Patient's blood glucose level is monitored by Continuous Glucose Monitoring System, and at least 10 times a day by vain blood sample. Ketones are measured 4 times a day by capillary blood sample. Urine samples are collected 3 times a day.

TABLE 10
Formulation E

| Materials Name | Quantity, g |
|---|---|
| Insulin | 0.3 |
| Silica R972 | 6 |
| Nutriose ®, | 20 |
| Beta-Cyclodextrin | 1.5 |
| Mannitol (Pearlitol) | 1 |
| L-Arginine | 1 |
| Olive oil | 15 |
| Oblepicha oil | 35 |
| Coconut oil | 15 |

Patients are followed 5 and 10 days following the first three days administration sessions and 5 days following the second (and last) three days administration session for drug safety evaluation. Table 11 summarizes the study procedure.

TABLE 11
study procedure
STUDY PROCEDURE

| | | | | | | |
|---|---|---|---|---|---|---|
| Transfer from | X | | | | | |
| pump to | | | | | | |
| injection | | | | | | |
| Connecting to | | X | | X** | | |
| glucose | | | | | | |
| monitoring | | | | | | |
| system | | | | | | |
| Disconnecting | | | | X* | | X |
| from glucose | | | | | | |
| monitoring | | | | | | |
| system | | | | | | |
| Oshadi | | | X | | X | |
| insulin/placebo | | | | | | |
| administration | | | | | | |
| Follow-up | | | | X | | X |
| visits | | | | | | |
| End of study | | | | | | X |
| Time line | 1 week prior | 1 day prior | Days | Days | Day | Day |
| | to drug | to drug | 1-4 | 9, 14 | 15-18 | 23 |
| | adminis- | adminis- | | | | |
| | tration | tration | | | | |
| Duration | 1 | 1 | 3 nights | 1 | 3 nights | 2 |
| | hour | hour | staying | hour | staying | hours |

*Day 9
**Day14

### Eligibility Criteria

### Inclusion Criteria:

1. Type 1 diabetes mellitus (according to ADA criteria) for more than 1 year.
2. Male/female 18 years old and older.
3. BMI≧18.5 and ≦25
4. Female of childbearing age must commit to avoid pregnancy and use contraception during the study.
5. Patients must understand and be willing to give written informed consent prior to any study procedures or evaluations and be willing to adhere to all study schedules and requirements.

Exclusion criteria included any other chronic or concurrent disease, except for controlled hypothyroidism.

### Sample Size:

10 evaluable Type 1 patients participate in the study. Additional patients may be enrolled to replace patients who discontinue study prematurely for reasons unrelated to safety or tolerance of oral insulin formulation or exacerbation of the underlying disease.

### Methods

### Screening Visit:

Patients are screened to determine their eligibility to be enrolled in the trial. Patients that use insulin pumps are transferred to basal and bolus doses based upon their current insulin pump regimen at least 1 week prior to study initiation. Generally, pump total basal rate may need to be increased by 20% to provide the dose of glargine (Lantus) basal insulin analog. Insulin to carb ratios and correction doses remain the same as utilized with pump therapy.

Day −1: Connecting the Continuous Glucose Monitoring device CGMS (±7 days).

Device is set to alarm when blood glucose level is <80 mg/dl and >350 mg/dl. The diet is adjusted to each patient to contain up to 90 g carbohydrate/day. In addition, patient is instructed to fast 10 h before coming to the Clinical research clinic (CRC).

Days 1-4: First Three Days Administration Session (±7 days)

Patient is administered with the insulin oral formulation or placebo 3 times a day during the staying in the CRC. In addition, patients are administered with half routine dose of pre-meal rapid acting insulin prior to meals, and half routine dose of long acting insulin at night. On the morning of day 4 patients return to their routine insulin regimen.

Patients are provided with 3 meals a day: Breakfast (≈450 calories/20 g carbohydrate); lunch (≈600 calories/30 g carbohydrate) and dinner (≈450 calories/20 g carbohydrate). Additional 20 g carbohydrate a day is provided according to patient needs.

Patients are encouraged to walk (modest walking) 30 min each day. No other exercises are permitted.

Blood glucose levels are monitored by a Continuous Glucose Monitoring System, at least 10 times a day by capillary blood sample (at morning, before and 2 h after meals, before bed time and twice during night sleeping). Ketones are measured 4 times a day by capillary blood sample before meals and before bed time. Assessments of real-time pharmacodynamic data are performed. In addition, urine samples are collected 3 times a day for C-peptide measurement.

Patients are provided with rescue rapid acting insulin to target of 100 mg/dl according to individual correction factor in the following hyperglycemic events:
Blood glucose level is >350 mg/dl prior to meal time;
Ketones >=1.0 mmol/L+blood glucose >250 mg/dl; will receive full correction factor bolus to target of 100 mg/dl if pre-meal or during the day and to target of 150 mg before bed time;
If Ketones >=0.6 mmol/L and blood glucose level >250 mg/dl a recheck should be performed in 1 hours;
Patients are provided with 30 gr fast acting carbs in the following hypoglycemic event:
Blood glucose level is ≦60 mg/dL; and
Blood glucose level ≦80 mg/dL with hypoglycemia symptoms.

A recheck is performed 20 minutes after carbhydrates ingestion.

Rapid acting insulin dose is reduced by 10% following the second hypoglycemic event, and 20% following the third hypoglycemic event.

On the morning of day 4 patients return to their routine insulin regimen. Safety lab tests and other baseline data (physical exam, ECG, Temp, BP, etc.) are performed prior to discharge (at 10:00 AM). In addition, urine samples are collected. Patients are encouraged to contact the investigator for any question or undesired effect. Insulin daily dose on that day will be recommended according to glucose levels to regular doses or to lower doses.

Day 9 and 14: Follow-up Visit (±7 days)

Patients are scheduled for a follow up visit on day 9 and 14. Follow-up includes blood pressure, temperature, weight measurements and ECG. Blood sampling for evaluation of insulin level in plasma is collected. In addition, urine samples are collected for C-peptide measurement. Patient is disconnected from the CGMS on day 9 and is reconnected on day 14. Patient is instructed to fast 10 h before coming to the CRC.

Days 15-18: Second Three Days Administration Session (±7 days)

Patients are admitted to the Clinical Research Center (CRC) at 7:00 AM of day 15 and remain there for 3 consecutive days. Study procedure is as described above for days 1-4, however, at this session the alternative drug (comparing to day 1-4, oral insulin formulation or placebo) is administrated.

Patients are provided with rescue rapid acting insulin or fast acting carbs if needed as in days 1-4.

On the morning of day 18 patients return to their routine insulin regimen. Patients that use insulin pumps will be transferred back to the insulin pump.

Safety lab tests and other baseline data (physical exam, ECG, Temp, BP, etc.) are performed prior to discharge (at 10:00 AM). In addition, urine samples are collected for C-peptide measurement. Patients are encouraged to contact the investigator for any question or undesired effect. Insulin daily dose on that day will be recommended according to glucose levels to regular doses or to lower doses.

Day 23: Follow-up Visits (±7 days)

Patients are scheduled for follow up visits on day 23. Follow-up includes blood pressure, temperature, and weight measurements, ECG and blood sampling for evaluation of insulin level in plasma will be collected. In addition, urine samples are collected for C-peptide measurement. The continuous Glucose Monitoring System is disconnected.

### Dose Adjustment of Insulin

Different patients use different insulin regimens to reduce blood glucose level. This study is double-blind, placebo-controlled, and thus the oral insulin dose administered is unchanged during the trial. If dose change is needed, the injectable insulin dose is adjusted. Dose adjustment of rapid acting subcutaneous insulin is based on pre-prandial glucose levels and carbohydrate intake during meal. Rescue of rapid acting insulin is also given if 2 measurements of night (pre-bedtime) glucose levels are above 300 mg/dL or if blood glucose level is above 350 mg/dl prior to meal time. Rapid insulin in those cases is given to target of 150 mg/dl. At night, bolus of insulin is given if blood glucose is >400 mg/dL without ketones or >300 mg/dL with ketones >1; if 300 with ketones 0.6 recheck in 1 hour.

If needed, the rapid acting insulin dose is reduced by 10% following the second hypoglycemic event and by 20% following the third hypoglycemic event.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A pharmaceutical composition for oral use comprising at least two bioactive proteins associated with glucose metabolism, selected from the group consisting of insulin, proinsulin and C-Peptide, in a delivery vehicle, adapted for oral administration that provides portal delivery of bioactive proteins, the delivery vehicle comprising an oil-based matrix comprising solid particulate matter suspended therein, wherein the particulate matter comprises a polysaccharide non-covalently associated with silica particles having a hydrophobic surface, wherein the polysaccharide and silica particles are non-covalently associated with the at least two bioactive proteins, and wherein the weight ratio of insulin to proinsulin is from about 25:1 to about 1:2;
the weight ratio of insulin to C-Peptide is from about 3:1 to about 1:2; and
the weight ratio of silica to the at least two bioactive proteins is from about 50:1 to about 1:1.

2. The pharmaceutical composition of claim 1, wherein the weight ratio of silica to insulin is from about 100:1 to about 2:1, wherein the weight ratio of silica to proinsulin is from about 200:1 to about 2:1 or wherein the weight ratio of silica to C-peptide is from about 200:1 to about 1:1.

3-4. (canceled)

5. The pharmaceutical composition of claim 1, wherein each of the bioactive proteins is non-covalently associated with said polysaccharide and silica particles.

6. (canceled)

7. The pharmaceutical composition of claim 1, wherein at least one bioactive protein associated with glucose metabolism is insulin.

8. The pharmaceutical composition of claim 1, the composition comprising insulin, proinsulin and C-Peptide non-covalently associated with the polysaccharide and silica particles, wherein the mixture of the bioactive proteins, silica particles and polysaccharide is suspended in the oil matrix.

9. (canceled)

10. The pharmaceutical composition of claim 1, wherein said polysaccharide is selected from the group consisting of starch, starch derivatives, amylopectin, glycogen, cyclodextrin and a combination thereof.

11-14. (canceled)

15. The pharmaceutical composition of claim 1, wherein the delivery vehicle further comprises an additional biopolymer selected from the group consisting of a polysaccharide and a high molecular weight structural protein, wherein said additional biopolymer is a linear biopolymer.

16-20. (canceled)

21. The pharmaceutical composition of claim 1, wherein said oil comprises a mixture of oils selected from natural vegetable oils and synthetic analogues thereof.

22. The pharmaceutical composition of claim 1, further comprising at least one additional component selected from the group consisting of antioxidants, amino acids, polypeptides, absorption enhancers, non-insulin glucose lowering drugs, blood pressure lowering drugs and combinations thereof.

23. (canceled)

24. The pharmaceutical composition of claim 22, wherein the antioxidant is selected from the group consisting of superoxide dismutase (SOD), glutathione peroxidase, a vitamin, glutathione, and an antioxidant mineral.

25. The pharmaceutical composition of claim 22, comprising at least one free amino acid, selected from the group consisting of arginine, leucine, isoleucine, histidine, phenylalanine and any combination and derivatives thereof.

26. The pharmaceutical composition of claim 22, wherein said pharmaceutical composition comprises at least one absorption enhancer selected from a medium chain fatty acid, a polyol or a combination thereof.

27. The pharmaceutical composition of claim 1, formulated in a form selected from the group consisting of liquid, solid, semi-solid, gel and microencapsulated forms.

28. The pharmaceutical composition of claim 27, formulated in a dosage form selected from the group consisting of a capsule, microcapsule, tablet, microencapsulated tablet, powder, suspension, paste and a combination thereof.

29. (canceled)

30. The pharmaceutical composition of claim 28, wherein the microencapsulated tablet comprises an excipient, which is present in the composition in a weight percent ranging from about 10% to about 80% of the total weight of the composition.

31-37. (canceled)

38. A pharmaceutical composition for oral use comprising at least two bioactive proteins associated with glucose metabolism, selected from the group consisting of insulin, proinsulin and C-Peptide, in a delivery vehicle, adapted for oral administration that provides portal delivery of bioactive proteins, wherein the weight ratio of insulin to proinsulin is from about 25:1 to about 1:2 and the weight ratio of insulin to C-Peptide is from about 3:1 to about 1:2.

39. The pharmaceutical composition of claim 38, wherein the delivery vehicle is selected from the group consisting of permeation enhancers, lipid delivery vehicles, liposomes, polymer matrices, polymeric microspheres, self-emulsifying drug delivery systems (SEDDS), molecules comprising alkoxy groups, non-ionic surfactants, nano-particle delivery systems and combinations thereof.

40-46. (canceled)

47. A method of treating diabetes in a subject in need thereof, comprising orally administering to said subject the pharmaceutical composition of claim 1.

48. The method of claim 47, wherein said diabetes is selected from the group consisting of: Type II diabetes, Type II diabetes related to obesity, gestational diabetes, Type I diabetes.

49. The method of claim 47, the method comprising administering said pharmaceutical composition instead of parenterally administered insulin or in combination with parenterally administered insulin.

50-52. (canceled)
